(19) United States
(12) Patent Application Publication
(10) Pub. No.: US 2015/0119558 A1
(43) Pub. Date: Apr. 30, 2015

(54) CHO-GMT RECOMBINANT PROTEIN EXPRESSION

(71) Applicant: AGENCY FOR SCIENCE, TECHNOLOGY AND RESEARCH, Singapore (SG)

(72) Inventor: Zhiwei Song, Singapore (SG)

(73) Assignee: AGENCY FOR SCIENCE, TECHNOLOGY AND RESEARCH, Singapore (SG)

(21) Appl. No.: 14/373,182

(22) PCT Filed: Jan. 18, 2013
(86) PCT No.: PCT/SG2013/000023

§ 371 Date: Jul. 18, 2014

(30) Foreign Application Priority Data
Jan. 20, 2012 (SG) 201200483-4
Sep. 24, 2012 (SG) 201207065-2

(51) Int. Cl. C07K 14/47

(52) U.S. Cl. CPC C07K 14/4727
USPC 530/395

(57) ABSTRACT

The present invention provides modified cells for producing proteins with modified glycosylation patterns. Proteins produced in such cells, and the use of such proteins in medicine, and particularly in the treatment of cancer, is also provided.

### FIELD OF THE INVENTION

The invention relates to the fields of biotechnology and molecular biology. The invention in particular relates to mammalian cell lines and their use in protein expression. The invention further relates to the use of such proteins in medicine.

### BACKGROUND TO THE INVENTION

Mucins are a family of heavily O-glycosylated secreted or membrane-associated large proteins produced by simple and glandular epithelia. A common feature shared by all mucin glycoproteins is the variable number of tandem repeat (VNTR) located in the central part of the molecule. These tandem repeats (TRs) are rich in serine, threonine and proline (for reviews see Thornton et al., 2008; Hattrup and Gendler, 2008). Human mucin 1 (MUC1) was the first mucin to be cloned and the best studied mucin to date. The MUC1 gene encodes a type I transmembrane protein with a single transmembrane domain and a C-terminal cytosolic tail. During maturation, MUC1 is cleaved into the N-terminal subunit and the C-terminal subunit. The C-terminal subunit contains the C-terminal 158-amino acids, which forms an extracellular domain, the transmembrane domain and a cytoplasmic tail. The size of the N-terminal subunit varies depending on the number of the TRs. After cleavage, the N-terminal subunit remains associated with the C-terminal subunit by interacting with the extracellular domain of the C-terminal subunit. The N-terminal 23-amino acids of MUC1 function as a signal peptide to target MUC1 to the cell surface and are removed during the process. The number of VNTR of the N-terminal subunit can vary between 25-100. Each TR contains identical twenty amino acids (HGVTSAPDTRPAPGSTAPPA) (SEQ ID NO: 1). The two S and three T residues in each repeat represent the five potential O-glycosylation sites. In addition to O-glycosylation, there are 5 potential N-glycosylation sites in the MUC1 molecule, 4 are located at the C-terminal end of the N-terminal subunit and one is located at the extracellular domain the of the C-terminal subunit (Thornton et al., 2008; Hattrup and Gendler, 2008).

Cell surface carbohydrates are characteristic of different stages of normal development and differentiation; distinct carbohydrates are expressed in tissue- and cell-specific patterns during those processes. The O-glycosylation pattern change of MUC1 is one of the best examples to illustrate this fact. In normal epithelial cells, MUC1 is expressed at low levels and restricted to the apical membranes of the epithelium to hydrate, protect and lubricate the surface. The TRs of MUC1 are O-glycosylated by highly branched complex carbohydrates. In comparison, MUC1 is highly overexpressed in adenocarcinomas, such as breast, ovarian and pancreatic cancers. In these cells, MUC1 is expressed over the entire cell surface and is no longer restricted to the apical surface. In addition, MUC1 is aberrantly glycosylated (for reviews see Taylor-Papadimitriou, 1999; Hollingsworth and Swanson, 2004; Tarp and Clausen, 2008; Kufe, 2009; Rachagani et al., 2009; Bafna et al., 2010). Instead of the highly branched complex carbohydrates, MUC1 expressed in cancer cells carries short immature O-glycans. Some of these short O-glycans are the Tn antigen (GalNAcα-O-Ser/Thr), Sialyl-Tn (STn) antigen (NeuAcα2-6GalNAcα-O-Ser/Thr) and T antigen (Galβ1-3GalNAcα-O-Ser/Thr) (Hull et al., 1989; Lioyd et al., 1996). Therefore, these short O-glycans are referred to as tumor-associated antigens and have been widely used as therapeutic targets and diagnostic markers (Vlad and Finn, 2004; Dube and Bertozzi, 2005; Brockhausen, 2006; Potapenko et al. 2010). In fact, the Tn, STn and T antigens were recognized as tumor-associated antigens long before the mucins were cloned (Springer, 1984; Springer, 1997). Other short O-glycans attached to the overexpressed MUC1 are sialyl-T (ST) antigen (NeuAcα2-3Galβ1-3GalNAcα-O-Ser/Thr) and disialyl-T (diST) antigen (NeuAcα2-3Galβ1-3[NeuAcα2-6]GalNAcα-O-Ser/Thr). They are not considered as tumor-associated antigens as they are also found in normal cells. As the precursors of more complex O-glycans, T and Tn epitopes are masked with other sugars in healthy adults. Therefore, they are un-detectable in healthy and benign-diseased tissues except in early embryonic stages. But they are expressed in about 90% of all carcinomas. Although T and Tn antigens are absent in normal adult tissues, all humans have anti-T and anti-Tn antibodies in our blood. This is likely the result of prior exposure to the commonly found Enterobacteriaceae that contain T and Tn epitopes. Chicks raised under germ-free conditions do not have anti-T and anti-Tn antibodies whereas chicks from the same hatch raised under ordinary conditions have these antibodies. Human infants produced high titers of anti-T and anti-Tn antibodies after being exposed to certain bacteria. Thus, T and Tn epitopes are “tumor-associated antigens” not “tumor-specific antigens”. Most anti-T antibodies in humans are IgM. In fact, anti-T IgM constitutes 7 to 14% of total IgM (Springer, 1984; Springer, 1997). However, no IgG antibodies to aberrantly O-glycosylated VNTR derived from MUC1 was detected in healthy individuals. IgG antibodies to Tn-VNTR were only detected in patients vaccinated with Tn-KLH or in sera from breast, ovarian, and prostate cancer patients (Wandall et al., 2010).

In contrast to the anti-T and anti-Tn antibodies which all of us have due to the humoral immune response to Enterobacteriaceae, there is no pre-existing cellular immune response to T and Tn antigens. Delayed-type hypersensitivity (DTH) is an inflammatory response that develops 24 to 48 hours after exposure to an antigen that the immune system recognizes as a foreign antigen. When the T epitope is used as the antigen in the assay, the DTH reaction to T antigen (DTHR-T) can be used to detect the cellular immune response to T antigen. Healthy individuals do not have a positive DTHR-T. Interestingly, DTHR-T was positive in most of the carcinoma patients. Carcinoma patients have both humoral and cellular immune response to T antigen whereas healthy people have only the humoral immune repose, but no cellular immune response, to T antigen (Springer, 1997).

In a recent report, the presence of auto-antibodies to aberrantly glycosylated MUC1 in early stage breast cancer has been associated with a better prognosis (Blixt et al., 2011). In that study, sera were collected in the 1970s and 1980s from a large cohort of breast cancer patients, patients with benign breast disease and healthy controls. In the clinical follow-up investigation, the presence and level of anti-aberrantly glycosylated MUC1 antibodies was found significantly higher in the sera from cancer patients compared with the controls. High levels of a subset of autoantibodies to the core3MUC1 (GlcNAcβ1-3GalNAc-MUC1) and STn MUC1 (NeuAcα2,6GalNAc-MUC1) glycoforms were significantly associated with reduced incidence and increased time to metastasis. Therefore, the association of strong antibody response with reduced rate and delay in metastases suggests that autoantibodies can affect disease progression (Blixt et al., 2011).

Aberrantly O-Glycosylated MUC1 has been a Target for Cancer Immunotherapy in Numerous Clinical Trials

MUC1 is highly immunogenic as most monoclonal antibodies raised against human cancer cell lines react with the TR region of MUC1 (Xing et al., 2001; Tang et al., 2008). The aberrant O-glycosylation of MUC1 results in the generation of cancer-associated antigens in two ways: (1) by exposure of protein backbone epitopes that are normally masked by the highly branched complex carbohydrates; (2) by changing the structure of the carbohydrate side chains attached to MUC1 to the well-known tumor-associated O-glycans, such as T, Tn and STn antigens. The loss of polarity of the epithelial cancer cells allows the deposition of these antigens on the whole surface of the cell which makes them more accessible to the immune system. In the last decade, numerous clinical trails have been carried out to test the anti-cancer properties of many MUC1-based vaccines (Richardson and Macmillan, 2008; Tang et al., 2008; Beatson et al., 2010). A few well-studied examples are discussed here.

STn-KLH (Theratope): STn is chemically linked to keyhole limpet haemocyanin (KLH) The STn antigen is expressed in about 30% of breast cancers (Julien and Delannoy, 2003; Miles and Papazisis (2003). As the expression of STn is highly restricted in normal tissues (Julien and Delannoy, 2003), this antigen has been considered a target for the development of anti-cancer vaccine (Tang et al., 2008). A synthetic STn-keyhole limpet haemocyanin (KLH) vaccine (Theratope), which consists of 3000 mol of the STn disaccharide conjugated to 1 mol of KLH, has been designed by the biotech company Biomira of Canada (now Oncothyreon, Seattle, USA; Rag upathi et al., 1999). Pre-clinical studies in mice showed that immunization with Theratope can induce STn-specific IgG. A phase II clinical study in breast cancer patients showed that potent STn-specific humoral responses can be induced following immunization with STn-KLH. However, in the follow-up phase III trial no benefit for Theratope-immunized patients compared to control group was shown (Holmberg et al., 2000; Finke et al., 2007). STn epitope alone without the MUC1 VNTR peptide backbone may not be specific enough to elicit strong immune response against aberrantly glycosylated MUC1 expressed on cancer cells. Nevertheless, studies using STn-KLH as a vaccine in animal models or in breast cancer patients are still on going in several labs (Gilewski et al., 2007; Julien et al., 2009).

BLP25 Liposome Vaccine (Stimuvax): Peptides Derived from the TR of MUC1 Mixed with Lipids

Another type of vaccine was based on unglycosylated peptides derived from MUC1 VNTR. In the early days, several groups have immunized mice with various synthetic peptides of different sizes derived from the VNTR of MUC1. While some peptides triggered humoral response others elicited T cell responses. A few triggered both humoral and T cell responses (Ding et al., 1993; Zhang et al., 1996; Soares et al., 2001). Recently, a vaccine that contains a 25-mer synthetic peptide based on the VNTR of MUC1 in a liposomal formulation, or BPL25 liposome vaccine (Stimuvax) has been developed and studied in clinical trials by Oncothyreon Inc. The BLP25 lipopeptide consists of a 25-amino acid sequence (STAPPAHGVTSAPDTRPAPGSTAPP)(SEQ ID NO; 2) that provides MUC1 specificity. It contains a palmitoyl lysine residue at the carboxy terminal to enhance the incorporation of the lipopeptide into the liposome particle. The vaccine consists of BLP25 lipopeptide, three lipids (cholesterol, dimyristoyl phosphatidylglycerol, and dipalmitoyl phosphatidylcholine) and immunoadjuvant monophosphoryl lipid A. The vaccine was granted fast-track status in September 2004 by the US FDA. At the end of 2008, Merck obtained the exclusive worldwide licensing rights from Oncothyreon Inc. for 13 million USD. Merck has been conducting. Phase III trials of Stimuvax to treat non-small cell lung cancer and breast cancer (Goldman and DeFrancesco, 2009).

### Mannosylated MUC1 Peptides

Antigen mannosylation is an effective approach to potentiate antigen immunogenicity, due to the enhanced antigen uptake and presentation by antigen presenting cells (APCs), particularly dendritic cells (DCs). Apostolopoulos and colleagues have been interested in targeting MUC1 to mannose-binding receptors on DCs. A peptide (five VNTRs) of MUC1 has been conjugated to yeast mannan to increase the uptake by DCs. They have shown that T1-type response which induces high cytotoxic T lymphocytes (CTLs) can be induced by conjugation of the antigen to the carbohydrate polymer mannan under oxidizing conditions (Apostolopoulos et al., 1995). The MUC1 peptide that was fused to GST conjugated to oxidized mannan was studied in several clinical trials (Loveland et al., 2006; Apostolopoulos et al., 2006; Tang et al., 2008). However, conventional chemical methods used to mannosylate antigens are not consistently reliable, due to the possibility of irreversible modification of immunodominant epitopes. Expression of soluble recombinant mannosylated proteins in yeast requires the introduction of glycosylation sites on the protein, as well as complicated characterization or optimization procedures. These issues need to be resolved in order to produce reliable mannosylated antigens.

### Tn-Glycosylated MUC1 Glycopeptide

The main problem of the above mentioned strategies is that the tumor-associated glycans and the MUC1-derived peptides are separated. When using unglycosylated MUC1-derived peptide as vaccines, the immune response are directed against the unglycosylated MUC1 only and not to the aberrantly glycosylated MUC1 which is expressed on the cancer cells. Attempts to generate strong humoral immunity to MUC1 by immunization with these unglycosylated peptides have generally failed partly because of tolerance. To overcome this problem, MUC1 TR glycopeptides with tumor-associated antigens were chemoenzymatically synthesized using a panel of recombinant human glycosyltransferases (Sørensen et al., 2006). MUC1 glycopeptides with different densities of Tn and STn glycoforms conjugated to KLH elicited the strongest antibody response reacting with MUC1 expressed in breast cancer cell lines. The elicited humoral immune response showed strong specificity for cancer cells suggesting that the glycopeptide design holds promise as a cancer vaccine. The elicited immune responses were directed against combined glycopeptide epitopes, and both peptide sequence and carbohydrate structures were important for the antigen.

### Recombinant MUC1 Produced by Wild Type CHO-K1 Cells

Recombinant MUC1 with 16 TRs has been produced and reported (Bäckström et al. 2003; Link et al. 2004). As this MUC1 was produced in wild type CHO-K1 cells, the O-glycans are ST and diST which are different from the tumor-associated antigen. Therefore, recombinant MUC1 produced by wild type CHO cells is chemically different from the MUC1 expressed on cancer cell surface.

### Limitations of Above-Described Studies

Each of the studies described above has its limitations. STn-KLH (Theratope) contains only the O-glycan but no MUC1 peptide. On the contrary, BLP25 liposome vaccine (Stimuvax) contains only the peptide and no glycans. MUC1 peptide conjugated to mannan seems to enhance antigen uptake by DCs. In vivo targeting of C-type lectin receptors on DCs is an effective strategy to increase efficacy of vaccines. However, this method has the same problem because it lacks the tumor-associated O-glycans attached to the MUC1 peptide. It has been shown that when humoral responses were elicited by unglycosylated MUC1 peptide, the antibodies were unable to recognize glycosylated MUC1 (von Mensdorff-Pouilly et al., 2000). The GSTA motif of the MUC1 20-amino acid tandem repeat has been shown to be a highly immunodominant epitope only when presented with immature short glycans (Tn/STn) (Tarp et al., 2007). Vlad et al. (2002) have shown that the O-glycans are not removed during processing of tumor antigen MUC1 glycopeptides by DCs. DCs endocytose glycopeptides, process them into smaller peptides and present them on MHC class II molecules without removing the carbohydrates. Resulting glycopeptide in the binding grove of MHC II triggers glycopeptide-specific CD4 T cells that can also respond to aberrantly glycosylated MUC1. Furthermore, O-glycosylation also affects intracellular processing of MUC1 by DC. One of the intracellular cleavage sites in MUC1 is between the Thr-Ser peptide bond within the 20-amino acid tandem repeat. However, if either amino acid residue is glycosylated, cleavage at this site is prevented (Hanisch et al., 2003). All these data suggest that glycosylated and unglycosylated MUC1 peptides represent different antigens and therefore MUC1 peptide used in the vaccines should be properly glycosylated. Chemoenzymatically synthesized Tn/STn MUC1 glycopeptides represent a step forward as they were able to elicit cancer-specific anti-MUC1 antibody responses (Sørensen et al., 2006). However, it is difficult to reach identical glycosylation patterns from batch to batch. New approaches are needed to produce the same vaccines consistently that closely mimic the structure of MUC1, both in peptide sequence and the pattern of glycosylation, in a more cost-effective manner.

### Mannose-Binding C-Type Lectins on Dendritic Cells (DCs) and Macrophages can Dramatically Enhance Antigen Uptake and Processing

DCs are the most potent antigen-presenting cells (APCs) and have a central role in directing the adaptive immune response. DCs are positioned at the external and internal surfaces of the body to specifically bind antigens and present them to lymphocytes in lymphoid organs. The uptake receptors on DCs can dramatically increase the efficiency for antigen capture and processing. Many of these uptake receptors are mannose-specific C-type lectins, such as langerin (CD207), DC-SIGN (CD209) and mannose receptor (CD206) (McGreal et al., 2005; Gazi et al., 2009; Keppler-Ross et al., 2010). Another type of APCs, macrophages, also uses mannose-specific C-type lectins as uptake receptors (Gazi et al., 2009). Therefore, mannosylation of vaccines has been an effective method to specifically target the antigens to DCs and macrophages for enhanced vaccine efficacy. As discussed above, yeast mannan has been conjugated to MUC1 peptides for enhanced immunogenicity. In addition to mannan, mannose-terminated N-glycans on recombinant proteins has also been shown to be recognized by mannose-specific C-type lectins and endocytosed by macrophages. One successful example comes from the treatment of the patients with Gaucher's disease with recombinant glucocerebrosidase that breaks down GlcCer within the macrophages. The recombinant glucocerebrosidase produced by wild-type CHO cells can only be captured efficiently by the macrophages when treated with sequential exoglycosidases until the mannose residues of the N-glycan (Man₃GlcNAc₂) are exposed (Friedman et al., 1999). Recombinant glucocerebrosidase produced by a CHO glycosylation mutant that has a dysfunctional GnT I gene contains mannose-terminated N-glycans (Man₅GlcNAc₂) (Goh et al., 2010). This product can also be specifically picked up by the C-type lectins on the surface of macrophages (Van Patten et al., 2007). We aim to produce MUC1 in a GnT I mutant CHO cell line that will result in MUC1 with mannose-terminated N-glycans. This feature could greatly enhance its efficacy as an anti-cancer vaccine.

MUC1 has been a target for cancer immunotherapy in several clinical trials. Theratope was developed by Biomira, Inc. as treatment for breast cancer. It is a synthetic vaccine in which STn disaccharides are chemically linked to a protein carrier, keyhole limpet haemocyanin (KLH). Theratope failed in phase III trials because STn epitope alone without the peptide backbone of the TRs was not specific enough to elicit strong immune response against aberrantly glycosylated MUC1 on cancer cells. BLP25 liposome vaccine (Stimuvax) contains unglycosylated peptides derived from TRs mixed with three lipids that act as adjuvants. Currently Merck is conducting a phase III trial on Stimuvax. A potential problem with Stimuvax is that unglycosylated MUC1 peptides are chemically different from glycosylated MUC1 expressed on cancer cells. The antibodies that bind the unglycosylated TRs will not be able to recognize the tumor-associated MUC1 with high affinity. Mannosylated MUC1 peptides have also been investigated as vaccines.

A peptide containing five TRs has been conjugated to yeast mannan to increase the uptake by dendritic cells (DCs). Mannose-binding C-type lectins on DCs and macrophages can dramatically enhance antigen uptake and processing. However, this MUC1 peptide also lacks the tumor-associated O-glycans. Gene therapy attempts have also been made by delivering the MUC1 cDNA into the patients. As the transgene will not be expressed in the cancer cells of the patients, the O-glycans on the MUC1 produced will be same as that of the normal cells. Therefore, this MUC1 vaccine will very likely be tolerated by the immune system. In summary, the problem with all these strategies is the failure of glycosylating the MUC1-derived peptides with tumor-associated short O-glycans. Chemoenzymatically synthesized multimeric Tn/STn MUC1 glycopeptides has elicited cancer-specific anti-MUC1 antibody responses and overridden tolerance, demonstrating the importance of O-glycans. However, chemical synthesis of Tn/STn MUC1 is very expensive and difficult to produce consistent products in large quantities.

### SUMMARY OF THE INVENTION

The present invention provides modified cells for producing proteins with modified glycosylation patterns. Proteins produced in such cells, and the use of such proteins in medicine, and particularly in the treatment of cancer, is also provided.

We have isolated two CHO glycosylated mutants: CHO-gmt1 (formerly MAR-11) and CHO-gmt2 (formerly MAR-1). CHO-gmt1 lacks functional CMP-sialic acid transporter and therefore the only O-glycan it can synthesize is the T antigen. CHO-gmt2 has a dysfunctional UDP-galactose transporter. Consequently, the O-glycans that CHO-gmt2 cells can produce are the Tn and STn antigens. We have also shown that most of CHO cells, if not all, that survive the cytotoxic RCA-I treatment have a mutated N-acetylglucosaminyltransferase I (GnT I) gene. As a result, the N-glycans produced by these mutants have the structure of Man₅GlcNAc₂, which can be specifically recognized by the mannose-binding C-type lectins on the surface of antigen presenting cells such as dendritic cells and macrophages. We have isolated cells that have mutated GnT I gene from CHO-gmt1 and CHO-gmt2 cells using RCA-1. The newly isolated mutants will produce proteins that are O-glycosylated with T or Tn and STn antigens and N-glycosylated with the Man₅GlcNAc₂ glycan. MUC1 produced in these double mutant lines possess enormous potentials as anti-cancer vaccines due to the presence of tumor-associated antigens attached to the VNTR region as well as mannose-terminated N-glycans which will allow for enhanced uptake by dendritic cells and macrophages. These recombinant N-terminal subunits of MUC1 will be investigated as anti-cancer vaccines in mouse models. Their efficacy in eliciting humoral and cellular immune responses will be investigated. Following positive results from the mouse models the next step is to produce the same recombinant proteins under GMP conditions for analysis in human cytotoxicity test.

The present invention relates to a glycoprotein that is expressed by a mammalian cell which has a mutated gene. The mammalian cell may have one or more of a mutated GnT I gene, a mutated CST gene, a mutated UGT gene and a mutated GFT gene.

Preferably, the mammalian cell comprises:
a) a mutated GnT I gene and a mutated CST gene;
b) a mutated GnT I gene and a mutated UGT gene; or
c) a mutated CST gene; or
d) a mutated UGT gene; or
e) a mutated GFT gene and a mutated CST gene; or
f) a mutated GFT gene.

The glycoprotein may comprise or consist of a Mucin, such as a Mucin which is aberrantly glycosylated in cancer cells. The Mucin may be a transmembrane Mucin. In particular, the recombinant protein may comprise, or consist of Mucin 1 (MUC1), or fragments thereof. Fragments of MUC1 may include the N terminal subunit of MUC1, and may include one or more, for example 1-10, 3-10, 11-20, 21-30, 31-40, 41-50, 51-60, 61-70, 71-80, 81-90, 91-100, 100 or more, 150 or more, or 200 or more tandem repeats.

The glycoprotein may be an antibody, antibody variant or antibody binding fragment. In such cases the mammalian cell preferably comprises a mutated GFT gene and a mutated CST gene.

The glycoproteins of the invention may have altered patterns of glycosylation. The altered glycosylation may be different to the glycosylation pattern associated with expression of the protein in a non-mutated mammalian cell. For example, a glycoprotein expressed in CHO cell with a mutation may have an altered pattern of glycosylation as compared to a glycoprotein expressed in a CHO cell which does not have that mutation.

The glycoprotein may have an altered pattern of glycosylation of N-glycans. The glycoprotein may comprise a mannose terminated glycan structure. The glycoprotein may comprise multiple mannose terminated glycan structures.

Additionally or alternatively, the glycoprotein may have an altered pattern of glycosylation of O-glycans. For example, the glycoprotein may have an altered pattern of sialylation and/or galactosylation. The glycoprotein may partially or entirely lack sialic acid, and/or may partially or entirely lack galactose.

In some embodiments the glycoprotein may comprise MUC1 or a fragment thereof that has T, Tn or Stn antigen glycans.

The mammalian cell may be a CHO cell, BHK cell, Vero cell, 293 cell, NS0 cell, 3T3 cell, COS-7 cell or PER C6 cell.

The invention also provides antibodies raised against the glycoproteins according to the invention.

The invention also provides glycoproteins (including antibodies), and antibodies there to, for use in medicine. The glycoprotein or antibody may be used in the treatment of cancer. The cancer may be an adenocarcinoma, such as breast cancer. The treatment may comprise administering to the patient a glycoprotein according to the invention, or an antibody there to. The treatment may comprise exposing cells (such as dendritic cells) isolated from the patient in need of treatment to a glycoprotein according to the invention, or an antibody thereto.

The invention further provides methods of medical treatment. The method may comprise administering a glycoprotein according to the invention, or an antibody thereto to a patient in need of such treatment. The patient may be suffering from cancer. The cancer may be an adenocarcinoma, such as breast cancer. Alternatively, the method may comprise obtaining cells from the patient and exposing those cells to a glycoprotein according to the invention, or an antibody thereto. The cells may be immune cells, such as dendritic cells.

The invention further provides glycoproteins, or antibodies thereto for use in the manufacture of a medicament for the treatment of cancer. The cancer may be an adenocarcinoma, such as breast cancer.

The invention further provides a mammalian cell. The mammalian cell may have one or more of a mutated GnT I gene, a mutated CST gene, a mutated UGT gene and a mutated GFT gene.

Preferably, the mammalian cell comprises:
a) a mutated GnT I gene and a mutated CST gene;
b) a mutated GnT I gene and a mutated UGT gene; or
c) a mutated CST gene; or
d) a mutated UGT gene; or
e) a mutated GFT gene and a mutated CST gene; or
f) a mutated GFT gene.

The mammalian cell may be a CHO cell, BHK cell, Vero cell, 293 cell, NS0 cell, 3T3 cell, COS-7 cell or PER C6 cell. In some cases, the mammalian cell is a cell of human origin. The cell may encode a heterologous glycoprotein, that is, a protein not normally produced by the cells from which the cell is derived. The heterologous glycoprotein may be encoded in the genome of the cell, or encoded by a vector contained within the cell.

The mammalian cell may express a recombinant protein. The glycoprotein may comprise or consist of a Mucin, such as a Mucin which is aberrantly glycosylated in cancer cells. The Mucin may be a transmembrane Mucin. In particular, the recombinant protein may comprise, or consist of Mucin 1 (MUC1), or fragments thereof. Fragments of MUC1 may include the N terminal subunit of MUC1, and may include one or more, for example 1 to 750, 1 to 500, 1 to 350, 1 to 200, 1 to 100 or 1 to 50 tandem repeats.

The invention also provides a culture of cells according to the invention.

The invention further provides a method of expressing a recombinant glycoprotein. In the method, the glycoprotein is expressed in a mammalian cell according to the invention. The glycoprotein may be encoded in the genome of the mammalian cell, or may be expressed from a vector in the cell.

Also provided herein are methods of treatment and agents for use in such methods. The methods involve administering glycoproteins produced by the methods described herein to patients. The glycoproteins may be administered the patient to be treated. Alternatively, antigen presenting cells such as dendritic cells, for example cells obtained from the patient to be treated, may be exposed to the glycoprotein ex vivo before being returned to the patient. The method may involve determining whether CD4+ and/or CD8+ responses specific for the glycoprotein have been stimulated in the patient.

In some cases, dendritic cells are obtained from a patient with a cancer and exposed to MUC1 produced in a cell described herein, such that it has an altered pattern of glycosylation relative to the glycosylation of MUC1 found in non-cancerous tissue. The dendritic cells may be exposed to a lysate of one or more modified CHO cells described herein, and which expresses MUC1. Following exposure to the CHO cell lysate, the dendritic cells are returned to the patient.

### DESCRIPTION OF PREFERRED EMBODIMENTS

The invention includes the combination of the aspects and preferred features described except where such a combination is clearly impermissible or expressly avoided.

The section headings used herein are for organizational purposes only and are not to be construed as limiting the subject matter described.

Aspects and embodiments of the present invention will now be illustrated, by way of example, with reference to the accompanying figures. Further aspects and embodiments will be apparent to those skilled in the art. All documents mentioned in this text are incorporated herein by reference.

### GnT I

The mammalian cells according to the invention may have a mutation in N-acetylglucoaminyltransferase I. For example modified cells may have a mutation in a gene that is, or is homologous to GenBank: AF343963.1 GI:14388960 (SEQ ID NO: 3).

We demonstrate that recombinant proteins of interest may be produced in cells lacking a functional GnT I gene and may comprise mannose terminated glycan structures. These results indicate that such cells have the potential to become a host cell line for producing proteins, comprising mannose-terminated N-glycans, including glycoprotein drugs and vaccines.

The mutation in GnT I in the mammalian cells according to the invention results in a loss of GnT I function. The GnT I function may be reduced or entirely absent.

Function of the GnT I gene in the cells according to the invention may be reduced as compared with the function of wild type GnT I, such as wild type human GnT I. In some cases the function is reduced relative to GnT I found in wild type CHO cells.

Expression of glycoproteins in such cells may be beneficial for expression of polypeptides which are to be targeted to cells with lectin receptors, for example, macrophages and dendritic cells. GnT I deficient cells made according to the methods described herein may be used for the production of recombinant proteins which have mannose-terminated glycan structures and are hence able to be taken up by macrophages.

This is in contrast to the normal complex-type N-glycans that also contain sialic acid and galactose residues using expression from other cells.

The use of GnT I deficient cells as host cells has a number of advantages. For example, as the recombinant expressed proteins of interest have mannose-terminated glycan structures, there is no need for enzymatic treatment to expose these.

Mammalian cells with a mutated GnT I have been previously described in WO2010/033085 and WO2011/119115.

The mutant GnT I cells according to the invention may have been identified, at least in part, by selection with RCA-I (Ricinus communis agglutinin I). The identification or selection protocol may be as set out in WO2010/033085 or WO2011/119115. Alternatively, the mutant gene may have been introduced into the mammalian cell by recombinant gene technology. The mammalian cell may be derived from, or otherwise comprise the same GnT I mutation as CI-10 cell line JW152.

The mutation in GnT I may comprise a substitution, deletion or addition. It may be a point mutation. The mutation may result in a premature stop codon.

### CST

The mammalian cells according to the invention may have a mutation in CMP-sialic acid transporter (CST). The CST may be, or be homologous to, NP_{—}001233684.1 GI:350537765 (SEQ ID NO: 4).

We demonstrate that recombinant proteins of interest may be produced in cells lacking a functional CST gene and may comprise different O-glycans to a recombinant glycoprotein produced in cells with an unmutated CST. These results indicate that such cells have the potential to become a host cell line for producing proteins with reduced sialylation, including glycoprotein drugs and vaccines.

The mutation in CST in the mammalian cells according to the invention results in a loss of CST function. The CST function may be reduced or entirely absent.

Function of the CST gene in the cells according to the invention may be reduced as compared with the function of wild type CST, such as wild type human CST. In some cases the function is reduced relative to CST found in wild type CHO cells.

Expression of glycoproteins in such cells may be beneficial for producing polypeptides with a similar or identical pattern of glycosylation to polypeptides with the same or very similar polypeptides produced by cancerous cells, for example adenocarcinoma cells. Cells with a mutated CST as described herein may be used for the production of proteins which have altered O-glycans compared to those produced by cells without a mutated CST. Such proteins may have reduced sialylation. The resulting proteins may comprise N-antigen type glycans, or be N-antigen glycoproteins.

### UGT

The mammalian cells according to the invention may have a mutation in UDP-galactose transporter (UGT). The UGT may be, or be homologous to, CBL95110.1 GI:296173022 (SEQ ID NO: 5).

We demonstrate that recombinant proteins of interest may be produced in cells lacking a functional UGT gene and may comprise different O-glycans to a glycoprotein produced in a cell with an unmutated UGT. These results indicate that such cells have the potential to become a host cell line for producing proteins with reduced galactosylation, including glycoprotein drugs and vaccines.

The mutation in UGT in the mammalian cells according to the invention results in a loss of UGT function. The UGT function may be reduced or entirely absent.

Function of the UGT gene in the cells according to the invention may be reduced as compared with the function of wild type UGT, such as wild type human UGT. In some cases the function is reduced relative to UGT found in wild type CHO cells.

Expression of glycoproteins in such cells may be beneficial for expression of polypeptides with a similar or identical pattern of glycosylation to polypeptides with the same or very similar polypeptides produced by cancerous cells, for example adenocarcinoma cells. Cells with a mutated UGT as described herein may be used for the production of proteins which have altered O-glycans compared to those produced by cells without a mutated CST. Such proteins may have reduced sialylation. The resulting proteins may comprise Tn-antigen or STn-antigen type glycans, or be Tn-antigen or STn-antigen glycoproteins.

### GFT

The mammalian cells according to the invention may have a mutation in GDP-fucose transporter (GFT). The GFT may be, or be homologous to, NP_{—}001233737.1 GI:350538845 (SEQ ID NO: 6).

The GDP-fucose transporter (abbreviated here as GFT, also known as Slc35c1) was first identified by genetic complementation analyses based on samples from CDG-IIc patients (8;9). Defects in this gene have been associated with leukocyte adhesion deficiency II (LAD-II), alias CDG-IIc. The amino acid sequence of GFT shows a high level of conservation with CMP sialic acid transporter (CST) and UDP-galactose transporter (UGT). It was predicted to have 10 transmembrane helices with both N- and C-termini in the cytosolic side, similar to the topology of CST which was experimentally established. However, elements that are critical for the localization and activity of GFT remain poorly understood.

We demonstrate that recombinant proteins of interest may be produced in cells lacking a functional GFT gene and may comprise reduced fucose in the N-glycans as compared to a recombinant protein produced in the presence of an unmutated GFT. These results indicate that such cells have the potential to become a host cell line for producing proteins with reduced fucose, including antibodies, glycoprotein drugs and vaccines.

The mutation in GFT in the mammalian cells according to the invention results in a loss of GFT function. The GFT function may be reduced or entirely absent.

Function of the GFT gene in the cells according to the invention may be reduced as compared with the function of wild type GFT, such as wild type human GFT. In some cases the function is reduced relative to GFT found in wild type CHO cells.

The mutation in GFT may be in the C terminal. The C terminal may be partially or entirely deleted.

Expression of glycoproteins in such cells may be beneficial for expression of polypeptides with a similar or identical pattern of glycosylation to polypeptides with the same or very similar polypeptides produced by cancerous cells, for example adenocarcinoma cells. Cells with a mutated GFT as described herein may be used for the production of proteins which have altered N-glycans that have reduced fucose as compared to those produced by cells without a mutated GFT. Such proteins may have reduced sialylation.

### Mucins

Mucins are a family of high molecular weight heavily glycosylated proteins produced by epithelial tissues. Overexpression of mucin proteins, especially MUC1 is associated with many types of cancer. Although some mucins are membrane bound due to the presence of a hydrophobic membrane spanning domain that favours retention in the plasma membrane, most mucins are secreted onto mucosal surfaces.

### MUC1 (Genbank Accession Number P15941.3, GI:296439295; SEQ ID NO: 7)

MUC1 is also known as Mucin-1, MUC-1, Breast carcinoma-associated antigen DF3, Carcinoma-associated mucin, Episialin, H23AG, PEMT, Peanut-reactive urinary mucin (PUM), Polymorphic epithelial mucin (PEM), Tumor-associated epithelial membrane antigen (EMA), Tumor-associated mucin and CD227.

Representative nucleic acid sequences of MUC1 include: Human pancreatic mucin mRNA (GenBank Accession Number J05582.1; GI:189598) (SEQ ID NO: 8), Homo sapiens mucin 1, cell surface associated (MUC1), transcript variant 1, mRNA (NCBI Reference Sequence: NM_{—}002456.4, GI:65301116) (SEQ ID NO: 9).

Representative amino acid sequences of MUC1 include: MUC1 (Homo sapiens) (GenBank Accession Number: CAA56734.1, GI:541680) (SEQ ID NO: 10), mucin-1 isoform 1 precursor (Homo sapiens) (NCBI Reference Sequence NP_{—}002447.4, GI:6501117) (SEQ ID NO: 11), mucin-isoform 2 precursor (Homo sapiens) (NCBI Reference Sequence NP_{—}001018016.1, GI:67189007) (SEQ ID NO:12), mucin-1 isoform 3 precursor (Homo sapiens) (NCBI Reference Sequence NP_{—} 001018017.1, GI:67189069) (SEQ ID NO:13).

Human mucin 1 (MUC1) is a heavily glycosylated transmembrane protein expressed on the apical surface of most epithelial tissues.

During maturation, MUC1 is cleaved into two subunits, an extracellular N-terminal subunit and a C-terminal transmembrane subunit. A unique feature of MUC1 is the variable-number of tandem repeats (VNTRs) that is located in the middle of the Nterminal subunit. Each tandem repeat (TR) contains identical 20 amino acids (HGVTSAPDTRPAPGSTAPPA) (SEQ ID NO:1). The Ser and Thr residues in the TR are O-glycosylation sites.

The N-terminal subunit also contains 4 N-glycosylation sites near its C-terminus. In normal epithelium, MUC1 is expressed at low levels and glycosylated with highly branched complex O-glycans.

However, in adenocarcinomas, such as breast, ovarian and pancreatic cancers, MUC1 is overexpressed in 90% of the cases. In addition, MUC1 expressed on cancer cells contains shortened O-glycans such as T antigen (Galβ1-3GalNAcα-O-Ser/Thr), Tn antigen (GalNAcα-O-Ser/Thr) and sialyl-Tn (STn) antigen (NeuAcα2-6GalNAcα-O-Ser/Thr) which are collectively called tumor-associated antigens. These glycans are not expressed in any adult tissues. MUC1 was recently recognized by the National Cancer Institute (USA) as one of the three most important tumor proteins for vaccine development. MUC1 is also overexpressed in 90% of the subset of breast cancer patients who are not responsive to tamoxifen or aromatase inhibitors, or the drug Herceptin. These so-called triple-negative tumors are extremely aggressive and difficult to treat. A vaccine directed against MUC1 has tremendous potential as a prophylactic vaccine or as a therapeutic vaccine for fighting breast cancers and other adenocarcinomas.

### Antibodies

Antibodies may be raised against glycoproteins produced by the cells according to the invention. Such antibodies may be useful in medicine, for example in the treatment of cancer. Such antibodies may be monoclonal or polyclonal. The antibodies may be specific to glycoproteins produced by the cells of the invention, such that they are able to distinguish between glycoproteins produced by the cells of the invention, and glycoproteins produced in wild type, or unmodified cells.

Suitable monoclonal antibodies can be prepared using methods well known in the art (e.g. see Köhler, G.; Milstein, C. (1975). “Continuous cultures of fused cells secreting antibody of predefined specificity”. Nature 256 (5517): 495; Siegel D L (2002). “Recombinant monoclonal antibody technology”. Schmitz U, Versmold A, Kaufmann P, Frank H G (2000); “Phage display: a molecular tool for the generation of antibodies—a review”. Placenta. 21 Suppl A: S106-12. Helen E. Chadd and Steven M. Chamow; “Therapeutic antibody expression technology,” Current Opinion in Biotechnology 12, no. 2 (Apr. 1, 2001): 188-194; McCafferty, J.; Griffiths, A.; Winter, G.; Chiswell, D. (1990). “Phage antibodies: filamentous phage displaying antibody variable domains”. Nature 348 (6301): 552-554; “Monoclonal Antibodies: A manual of techniques”, H Zola (CRC Press, 1988) and in “Monoclonal Hybridoma. Antibodies: Techniques and Applications”, J G R Hurrell (CRC Press, 1982). Chimaeric antibodies are discussed by Neuberger et al (1988, 8th International Biotechnology Symposium Part 2, 792-799)).

In certain embodiments of the invention the glycoprotein expressed by the cells of the invention is an antibody or antibody fragment. In these embodiments, the cell preferably comprises a mutated GFT.

Fragments of antibodies, such as Fab and Fab₂ fragments may also be used as can genetically engineered antibodies and antibody fragments. The variable heavy (V_{H}) and variable light (V_{L}) domains of the antibody are involved in antigen recognition, a fact first recognised by early protease digestion experiments. Further confirmation was found by “humanisation” of rodent antibodies. Variable domains of rodent origin may be fused to constant domains of human origin such that the resultant antibody retains the antigenic specificity of the rodent parented antibody (Morrison et al (1984) Proc. Natl. Acad. Sd. USA 81, 6851-6855).

That antigenic specificity is conferred by variable domains and is independent of the constant domains is known from experiments involving the bacterial expression of antibody fragments, all containing one or more variable domains. These molecules include Fab-like molecules (Better et al (1988) Science 240, 1041); Fv molecules (Skerra et al (1988) Science 240, 1038); single-chain Fv (ScFv) molecules where the V_{H} and V_{L} partner domains are linked via a flexible oligopeptide (Bird et al (1988) Science 242, 423; Huston et al (1988) Proc. Natl. Acad. Sd. USA 85, 5879) and single domain antibodies (dAbs) comprising isolated V domains (Ward et al (1989) Nature 341, 544). A general review of the techniques involved in the synthesis of antibody fragments which retain their specific binding sites is to be found in Winter & Milstein (1991) Nature 349, 293-299.

By “ScFv molecules” we mean molecules wherein the V_{H} and V_{L} partner domains are covalently linked, e.g. directly, by a peptide or by a flexible oligopeptide. Fab, Fv, ScFv and dAb antibody fragments can all be expressed in and secreted from E. coli, thus allowing the facile production of large amounts of the said fragments.

Whole antibodies, and F(ab′)₂ fragments are “bivalent”. By “bivalent” we mean that the said antibodies and F(ab′)₂ fragments have two antigen combining sites. In contrast, Fab, Fv, ScFv and dAb fragments are monovalent, having only one antigen combining site. Synthetic antibodies which bind to the glycoproteins of the invention may also be made using phage display technology as is well known in the art (e.g. see “Phage display: a molecular tool for the generation of antibodies—a review”. Placenta. 21 Suppl A: S106-12. Helen E. Chadd and Steven M. Chamow; “Phage antibodies: filamentous phage displaying antibody variable domains”. Nature 348 (6301): 552-554).

### Therapy

Glycoproteins of the present invention may be used in medicine. In certain embodiments, the glycoproteins of the invention are useful in the treatment of tumours and cancer in animals in need of treatment thereof. Preferably, the animal undergoing treatment is a human patient in need of such treatment. Preferably the cells are mammalian cells, more preferably human cells.

Glycoproteins of the invention may be formulated as pharmaceutical compositions for clinical use and may comprise a pharmaceutically acceptable carrier, diluent or adjuvant. The composition may be formulated for topical, parenteral, intravenous, intratumoral, intramuscular, intrathecal, intraocular, subcutaneous, oral, inhalational or transdermal routes of administration which may include injection. Injectable formulations may comprise the selected compound in a sterile or isotonic medium.

Administration is preferably in a “therapeutically effective amount”, this being sufficient to show benefit to the individual. The actual amount administered, and rate and time-course of administration, will depend on the nature and severity of the disease being treated. Prescription of treatment, e.g. decisions on dosage etc, is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 20th Edition, 2000, pub. Lippincott, Williams & Wilkins.

The term “treatment” as used herein pertains generally to treatment and therapy, in which some desired therapeutic effect is achieved, for example, the inhibition of the progress of the condition, and includes a reduction in the rate of progress, a halt in the rate of progress, amelioration of the condition, and the cure of the condition. Treatment as a prophylactic measure (i.e. prophylaxis) is also included.

Alternatively, targeting therapies may be used to deliver the active agent more specifically to certain types of cell, by the use of targeting systems such as antibody or cell specific ligands. Targeting may be desirable for a variety of reasons; for example if the agent is unacceptably toxic, or if it would otherwise require too high a dosage, or if it would not otherwise be able to enter the target cells.

A composition may be administered alone or in combination with other treatments, either simultaneously or sequentially dependent upon the condition to be treated.

### APC Therapy

In some embodiments of the invention, cells that have been obtained from the patient in need of treatment are exposed to a glycoprotein expressed by a cell according to the invention outside of the body, i.e. ex vivo. For example, the cells may be pulsed with the glycoprotein. The cells may be pulsed with a plurality of glycoproteins expressed in different modified cells described herein. The cells obtained from the patient may be immune cells, for example dendritic cells or monocytes. Following exposure to the glycoprotein, the cells may be returned to the patient in need of treatment thereof. The cells isolated from the patient are expanded ex vivo, before and/or after exposure to the glycoprotein.

In certain embodiments of the invention, Antigen Presenting Cells (APCs) such as dendritic cells are exposed in vitro to the glycoproteins described herein. The APCs may be pulsed with one or more glycoproteins. The APCs may be exposed to a lysate of one or more of the mammalian cells described herein which contains the glycoprotein, or to an isolated and/or purified glycoprotein. The lysate may be a lysate of a cell in which the glycoprotein was expressed. The APCs may have been derived from the patient to be treated, for example isolated from a sample of blood obtained from the patient.

Methods of APC therapy are known in the art. APCs may be obtained from a patient to be treated and cultured in vitro in the presence of one or more cytokines. They may be exposed to the glycoproteins in the presence of one or more adjuvant agents. The APCs may be exposed to more than one glycoprotein. For example, the APC may be exposed to a plurality of different purified glycoproteins, or a lysate from a cell expressing one or more glycoproteins, or a mixture of lysates from more than one type of cell, each expressing one or more glycoproteins. The glycoprotein may be a peptide fragment of a cellular glycoprotein.

APCs that have been exposed to glycoprotein may be reintroduced into the patient by any suitable route, for example, systemic, parenteral, intratumoral or other administration route. They may be reintroduced in the presence of one or more adjuvant agents, and/or pharmaceutically acceptable carriers and/or excipients. Optionally, the APCs may be administered in conjunction with another therapy, such as a chemotherapeutic agent.

Antigen specific CD4+ and CD8+ cells may be generated in the patient in response to the treatment.

### Patient

The patient to be treated may be any animal or human. The patient is preferably a mammal, more preferably a human patient. The patient may be a non-human mammal. The patient may be male or female.

In some cases the patient suffers from cancer. Cancers treatable by the methods of the invention include breast, colon, ovarian, lung and pancreatic cancer. It may be a carcinoma such as an adenocarcinoma. The cancer may be a metastatic cancer.

A “cancer” can comprise any one or more of the following: acute lymphocytic leukemia (ALL), acute myeloid leukemia (AML), adrenocortical cancer, anal cancer, bladder cancer, blood cancer, bone cancer, brain tumor, breast cancer, cancer of the female genital system, cancer of the male genital system, central nervous system lymphoma, cervical cancer, childhood rhabdomyosarcoma, childhood sarcoma, chronic lymphocytic leukemia (CLL), chronic myeloid leukemia (CML), colon and rectal cancer, colon-cancer, endometrial cancer, endometrial sarcoma, esophageal cancer, eye cancer, gallbladder cancer, gastric cancer, gastrointestinal tract cancer, hairy cell leukemia, head and neck cancer, hepatocellular cancer, Hodgkin's disease, hypopharyngeal cancer, Kaposi's sarcoma, kidney cancer, laryngeal cancer, leukemia, leukemia, liver cancer, lung cancer, malignant fibrous histiocytoma, malignant thymoma, melanoma, mesothelioma, multiple myeloma, myeloma, nasal cavity and paranasal sinus cancer, nasopharyngeal cancer, nervous system cancer, neuroblastoma, non-Hodgkin's lymphoma, oral cavity cancer, oropharyngeal cancer, osteosarcoma, ovarian cancer, pancreatic cancer, parathyroid cancer, penile cancer, pharyngeal cancer, pituitary tumor, plasma cell neoplasm, primary CNS lymphoma, prostate cancer, rectal cancer, respiratory system, retinoblastoma, salivary gland cancer, skin cancer, small intestine cancer, soft tissue sarcoma, stomach cancer, stomach cancer, testicular cancer, thyroid cancer, urinary system cancer, uterine sarcoma, vaginal cancer, vascular system, Waldenstrom's macroglobulinemia and Wilms' tumor.

Cancers may be of a particular type. Examples of types of cancer include astrocytoma, carcinoma (e.g. adenocarcinoma, hepatocellular carcinoma, medullary carcinoma, papillary carcinoma, squamous cell carcinoma), glioma, lymphoma, medulloblastoma, melanoma, myeloma, meningioma, neuroblastoma, sarcoma (e.g. angiosarcoma, chrondrosarcoma, osteosarcoma).

### Proteins, Fragments and Derivatives

Whilst components used in the methods of the present invention may comprise full-length protein sequences, this is not always necessary. As an alternative, homologues, mutants, derivatives or fragments of the full-length polypeptide may be used.

Derivatives include variants of a given full length protein sequence and include naturally occurring allelic variants and synthetic variants which have substantial amino acid sequence identity to the full length protein.

Protein fragments may be up to 10, 20, 30, 40, 50, 60, 70, 80, 90, 100 or 150 amino acid residues long. Minimum fragment length may be 3, 4, 5, 6, 7, 8, 9, 10, 11, 12, 13, 14, 15, 16, 17, 18, 19, 20 or 30 amino acids or a number of amino acids between 3 and 30.

Mutants may comprise at least one modification (e.g. addition, substitution, inversion and/or deletion) compared to the corresponding wild-type polypeptide. The mutant may display an altered activity or property, e.g. binding. Molecular biology techniques suitable for producing genetic mutations according to the invention in cells are well known in the art, such as those set out in Sambrook et al., Molecular Cloning: A Laboratory Manual, New York: Cold Spring Harbor Press, 1989.

Mutations may occur in any of the genes or sequences discussed herein and components containing such fragments may serve the purpose of modulating the activity of the mutant to restore, completely or partially the activity of the wild type polypeptide.

Derivatives may also comprise natural variations or polymorphisms which may exist between individuals or between members of a family. All such derivatives are included within the scope of the invention. Purely as examples, conservative replacements which may be found in such polymorphisms may be between amino acids within the following groups:
(i) alanine, serine, threonine;
(ii) glutamic acid and aspartic acid;
(iii) arginine and leucine;
(iv) asparagine and glutamine;
(v) isoleucine, leucine and valine;
(vi) phenylalanine, tyrosine and tryptophan.

Derivatives may also be in the form of a fusion protein where the protein, fragment, homologue or mutant is fused to another polypeptide, by standard cloning techniques, which may contain a DNA-binding domain, transcriptional activation domain or a ligand suitable for affinity purification (e.g. glutathione-S-transferase or six consecutive histidine residues.

Methods for expressing a protein of interest from a mammalian cell are known in the art. A gene encoding the glycoprotein of interest may be inserted into the genome of a mammalian cell, or introduced on a vector. The DNA of the gene is transcribed to messenger RNA which is then translated into polypeptide chains which are ultimately folded into proteins.

Following culture of cells that express glycoprotein, the glycoprotein is preferably isolated. Any suitable method for separating proteins from cell culture known in the art may be used. In order to isolate a protein of interest from a culture, it may be necessary to first separate the cultured cells from media containing the protein of interest. If the protein of interest is secreted from the cells, the cells may be separated from the culture media that contains the secreted protein by centrifugation. If the protein of interest collects within the cell, it will be necessary to disrupt the cells prior to centrifugation, for example using sonification, rapid freeze-thaw or osmotic lysis. Centrifugation will produce a pellet containing the cultured cells, or cell debris of the cultured cells, and a supernatant containing culture medium and the protein of interest.

It may then be desirable to isolate the protein of interest from the supernatant or culture medium, which may contain other protein and non-protein components. A common approach to separating protein components from a supernatant or culture medium is by precipitation. Proteins of different solubilities are precipitated at different concentrations of precipitating agent such as ammonium sulfate. For example, at low concentrations of precipitating agent, water soluble proteins are extracted. Thus, by adding different increasing concentrations of precipitating agent, proteins of different solubilities may be distinguished. Dialysis may be subsequently used to remove ammonium sulfate from the separated proteins.

The protein fragments or derivatives may be fragments or derivatives of a Mucin, such as a Mucin that is aberrantly glycosylated in cancer cells. The Mucin may be one that is a transmembrane Mucin. In particular, they may be fragments or derivatives of MUC1.

The fragments or derivatives may contain the N terminal subunit of MUC1. They may contain one or more tandem repeat sequences. Each tandem repeat (TR) may contain 20 amino acids and have the sequence HGVTSAPDTRPAPGSTAPPA (SEQ ID NO: 1). The Ser and Thr residues in the TR may be the O-glycosylation sites. In some cases, the fragment may comprise the sequence STAPPAHGVTSAPDTRPAPGSTAPP (SEQ ID NO:2). The peptide may comprise 1 to 500, 1 to 250, 1 to 100, 1 to 50, 1 to 35, 1 to 30, 1 to 25, 1 to 20, 1 to 15, 1 to 10, 1 to 5, or 2or more TRs. They may contain 1-10, 3-10, 11-20, 21-30, 31-40, 41-50, 51-60, 61-70, 71-80, 81-90, 91-100, 100 or more, 150 or more, or 200 or more TRs.

The proteins and peptides may be expressed as a fusion protein with an Fc domain of an immunoglobulin, such as IgG. Fusion to Fc may improve the stability and deliverability of the protein or peptide.

### BRIEF DESCRIPTION OF THE FIGURES

Embodiments and experiments illustrating the principles of the invention will now be discussed with reference to the accompanying figures in which:

FIG. 1. N- and O-glycans attached to MUC1 produced by wild type and mutant CHO cells. The mutant cells produce mannose-terminated N-glycans to enhance uptake by dendritic cells and tumor-associated O-glycans to trigger cancer specific immune responses. A, glycans produced by different CHO cells. B, N-terminal subunit of MUC1 produced by different CHO cell.

FIG. 2. Genetic analyses of CHO-gmt1 (A) and CHO-gmt2 (B). CHO-gmt1 lacks CMP-sialic acid transporter (CST) activity. CHO-gmt2 lacks UDP-galactose transporter (UGT) activity.

FIG. 3. Genetic analyses of CHO-gmt6 (A) and CHO-gmt7 (B). CHO-gmt6 lacks both CMP-sialic acid transporter (CST) activity and N-acetylglucosaminyltransferase I (GnT I) activity. CHO-gmt7 lacks both UDP-galactose transporter (UGT) activity and GnT I activity.

FIG. 4. Recombinant MUC1-Fc produced by CHO-K1 and different CHO glycosylation mutant cells were analyzed by Western blot using anti-MUC1

FIG. 5. Design of ZFNs targeting the GFT gene in CHO cells. (A) A DNA sequence in the coding region of Chinese hamster GFT gene that is ideal for targeting by ZFNs (SEQ ID NOs:14 & 15). Note that the left finger binding site and the right finger binding site are separated by 6 bps. (B) Zinc fingers were designed based on published literature. Each ZFN contains 4 fingers (SEQ ID NOs 16-23). (C) Mutations at the targeted site were identified in different clones, including deletion (SEQ ID NOs: 24-31) and insertion mutations. (SEQ ID NOs: 32 & 33). Both alleles of the GFT gene in clone E were mutated by the ZFNs. It has been named CHO-gmt5.

FIG. 6. Lectin staining and MALDI-TOF characterization of glycan structures of wild-type and mutant CHO cells. (A) CHO-K1, CHO-gmt1, and CHO-gmt5 cells were seeded on glass coverslips, cultured overnight before being fixed and permeabilized. Terminal galactose residues were detected using FITC-conjugated PNA (colored green). Fucose residues were detected using biotinylated ML and AlexaFluor 647-conjugated streptavidin (pseudo-colored red). Nuclei were stained by Hoechst 33342 and colored blue. (B) N-glycans isolated from recombinant EPO-Fc produced in CHO-gmt1 and CHO-gmt5 cells were analyzed by MALDI-TOF. The N-glycans produced by CHO-gmt1 were mostly fucosylated, and the dominant species were the asialo, core-fucosylated galactosyl biantennary, triantennary and tetraantennary glycans. However, the N-glycans produced by CHO-gmt5 were the asialo, afucosylated galactosyl bi-antennary, tri-antennary and tetra-antennary glycans.

FIG. 7. Localization of GFT within the Golgi. HA-tagged human GFT was transiently transfected into HeLa cells and GFT was detected by a monoclonal anti-HA antibody (colored green). Golgi compartments were stained by antibodies specific for defined Golgi markers, GM130 (cis-Golgi), ManII (medial-Golgi), B4GalT1 (trans-Golgi) and TGN46 (trans Golgi network, or TGN). All Golgi markers were colored red. Nuclei were stained by Hoechst 33342 and colored blue. The boxed areas in the merged images were enlarged and shown on the right. Scale bar: 20 μm.

FIG. 8. Charts showing FACS analysis of cells. (A) About 12000 cells were collected out of 3.5 million cells processed through the sorter, these cells were cultured and grown for ˜2 weeks before subjecting to a second round of sorting (B). Further FACS analysis of the resultant enriched pool showed that most of the cells were AAL-ve cells (C). To confirm that the AAL-ve phenotype was due to the lack of functional GDP-fucose transporter gene, the enriched cells were also separately transfected with a construct that expresses the GDP-fucose transporter and analyzed by FACS (D).

FIG. 9. Table showing sequencing results of three CHO-gmt3 clones (SEQ ID NOs: 34-40). Bold and underline indicates target region for ZFN. Shading indicates insertion mutation, − indicates deletion mutation.

FIG. 10. MUC1 expression in CHO cells. MUC1 was expressed in CHO cells and stained with an antibody. FM 4-64FX is a plasma membrane stain.

### DETAILED DESCRIPTION OF THE INVENTION

The details of one or more embodiments of the invention are set forth in the accompanying description below including specific details of the best mode contemplated by the inventors for carrying out the invention, by way of example. It will be apparent to one skilled in the art that the present invention may be practiced without limitation to these specific details.

### Example 1

### CHO Glycosylation Mutants can be Used to Produce Recombinant MUC1 with Tumor-Associated O-Glycans

Chinese hamster ovary (CHO) cells have been the main host cells for producing recombinant glycoproteins in the biotechnology industry. The N- and O-glycosylation in CHO cells has been a major research focus for many years. The O-glycosylation pathways can be complex and cell type specific in different mammalian cells (Tarp and Clausen, 2008). However, the O-glycosylation in CHO cells is very simple with a core 2 to core 1 shift. The O-glycans on the recombinant erythropoietin (EPO) produced by CHO cells only existed in two forms: ST and diST (Hokke et al., 1995). Recombinant MUC1 produced by CHO cells also contained mainly ST and diST (Bäckström et al., 2003; Olson et al., 2005; Rughetti et al., 2005). A detailed analysis revealed that the O-glycans attached to recombinant MUC1 produced in CHO cells contained 85% ST, 13% diST and only 2% T (Rughetti et al., 2005). The total O-glycans derived from CHO cells grown in suspension and CHO cells grown in monolayer both contained ST and diST (North et al., 2010).

Numerous CHO glycosylation mutants have been isolated and characterized by several labs. Stanley and colleagues reported the Lec (for lectin resistant) series of CHO glycosylation mutants (Patnaik and Stanley P, 2006). Unfortunately, these cells do not grow in serum-free medium in suspension cultures. As a result, although the Lec mutants played important roles in elucidating the biological functions of protein glycosylation and possible applications of these mutants in biotechnology industry, they have not been utilized in large scale production of any recombinant proteins. Using cytotoxic lectins, mainly Maackia amurensis agglutinin (MAA) and Ricinus communis agglutinin-I (RCA-I, or RCA-120), we have isolated several CHO glycosylation mutants from the wild-type CHO-K1 cells (Lim et al., 2008; Goh et al., 2010). These mutants can easily be adapted in serum-free medium and cultured in suspension in bioreactors. The growth rate and final viable cell density reached by these mutants are comparable to wild-type CHO-K1 cells. Therefore, our CHO mutants have the potential to be the host cells for large scale production of recombinant proteins. The following three existing CHO glycosylation mutants characterized in this lab are relevant to this proposal and will be described in more details.

### CHO-gmt1

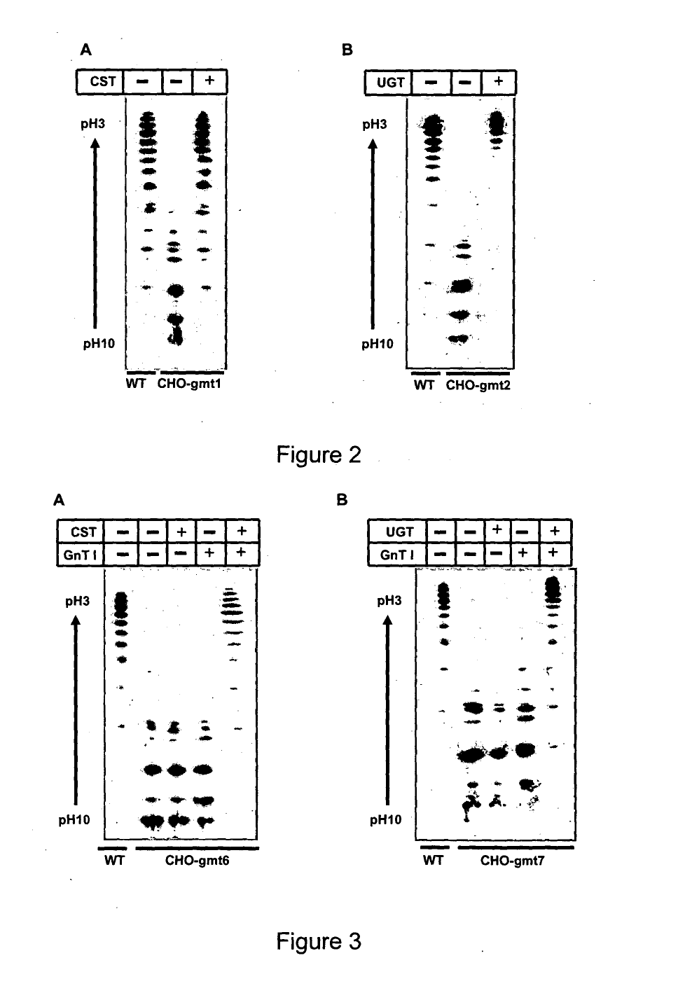

CHO-gmt1 (formerly MAR-11) has been characterised (Lim et al., 2008). The total cell surface sialic acid on CHO-gmt1 is lower than that on a previously reported CHO mutant line, Lec2. Biochemical analyses indicate that recombinant glycoproteins produced by these cells lack sialic acid. Genetic test suggested that CHO-gmt1 cells lacked CMP-sialic acid transporter (CMP-SAT) activity (FIG. 2A). Molecular cloning of CMP-SAT cDNA from CHO-gmt1 cells revealed a C to T point mutation which results in a premature stop codon. As a result, CHO-gmt1 cells express a truncated version of CMP-SAT which contains only 100 amino acids, in comparison to the normal CMP-SAT which contains 336 amino acids (Lim et al., 2008). The predicted O-glycans produced in CHO-gmt1 cells will be mainly T antigen which is tumor-associated antigens.

### CHO-gmt2

CHO-gmt2 was also isolated by MAA. CHO-gmt2 has a mutated UDP-galactose transporter (UDP-GalT) gene (FIG. 2B). The G538C mutation in the open reading frame results in a A180P mutation in the UDP-GalT protein. With a dysfunctional UDP-GalT, CHO-gmt2 cannot transport UDP-Gal into the Golgi apparatus. As a result, O-glycans produced in CHO-gmt2 cells will be Tn and STn which are also tumor-associated antigens. It has been shown recently that Tn-based vaccines may directly target DCs and develop potent antibody response in the absence of adjuvant (Freire et al., 2010).

### CHO-gmt4

CHO-gmt4 (formerly JW152) was isolated by RCA-I and has been characterised (Goh et al., 2010). Complementation tests revealed that all the RCA-I-resistant mutants possessed a dysfunctional GnT I gene (Goh et al., 2010). Protein O-glycosylation in these cells should not be affected. However, the N-glycosylation is prematurely terminated at the Man₅GlcNAc₂ stage due to the lack of GnT I. We have shown that recombinant EPO produced in these cells carries Man₅GlcNAc₂ glycan and some fucosylated Man₅GlcNAc₂ glycan. A small amount of Man₄GlcNAc₂ was also attached to the recombinant EPO. As described earlier, proteins that carry mannose-terminated N-glycans can be targeted to DCs and macrophages via their mannose-binding C-type lectins. A surprising finding during the course of this work was that all the CHO cells that survived the RCA-I treatment carry a mutated GnT I gene. A possible explanation for this finding is that RCA-I may bind all different N-glycans, although with different affinity, except Man₅GlcNAc₂ N-glycan. Therefore, all the cells survived RCA-I must have a dysfunctional GnT I. In our lab, treating CHO cells with RCA-I has become a simple method to isolate cells with mutated GnT I gene. We have filed two international patent applications for this discovery (Patent I: Pub. No.: US 2011/0177555 A1. Title: GnT I Mutant CHO Cell Lines; Patent II: U.S. Patent Application 61/317,369 Title: Method of Producing Mannose-terminated Recombinant Proteins).

### CHO-gmt6 and CHO-gmt7

We have isolated two novel CHO glycosylated mutants that have been named CHO-gmt6 and CHO-gmt7 respectively. CHO-gmt6 cells lack functional CMP-sialic acid transporter (CST) and GnT I (FIG. 3A). CHO-gmt7 cells lack UDP-galactose transporter (UGT) and GnT I (FIG. 3B). As a result, the N- and O-glycans produced in CHO-gmt6 and CHO-gmt7 cells are different from that produced in wild type CHO cells. The N-glycans produced by these two mutants are Man₅GlcNAc₂. This mannose-terminated N-glycan can be specifically recognized by the mannose-binding receptors on DCs and macrophages and therefore enhances the antigenicity of the protein. The O-glycans produced by CHO-gmt6 are T antigens whereas the O-glycans produced by CHO-gmt7 are Tn and STn antigens.

CHO-gmt6 and CHO-gmt7 will be used as host cells to produce a smaller version of the N-terminal subunit of MUC1 as anti-cancer vaccine. This recombinant N-terminal subunit of MUC1 will have 5-10 TRs. The T, Tn and STn O-glycans attached to the MUC1 produced by CHO-gmt6 and CHO-gmt7 can trigger tumor-specific immune response in the host as their O-glycans are the same as those attached to the MUC1 on tumor cells. The mannose-terminated N-glycan on this recombinant MUC1 will increase it immunogenicity by enhancing the uptake by DCs and macrophages.

### Expression of Recombinant MUC1 N-Terminal Subunit in Newly Isolated CHO Glycosylation Mutants, CHO-Gmt6 and CHO-Gmt7.

Expression constructs will be generated to express a modified N-terminal subunit of MUC1. The amino acid sequence of the N-terminal subunit of MUC1 will remain the same except the number of the VNTR will be reduced to 5-10. The cDNA will encode 497 amino acids. During maturation, a signal peptide of 23 amino acids at the N-terminus will be removed. The secreted mature proteins will contain 474 amino acids. There will be 50 potential O-glycosylation sites in the 10 TR region and 4 N-glycosylation sites at the C-terminal region. In the future, the number of TRs in the construct may increase or decrease. The protein will be transiently expressed in CHO-K1 cells and CHO-gmt6 and CHO-gmt7 cells. The presence of the recombinant protein in conditioned media will be probed by anti-MUC1 antibodies. Stably transfected clones that express high levels of recombinant MUC1 N-terminal subunit will be isolated and banked for large scale production.

### Purification of Recombinant MUC1 N-Terminal Subunit Expressed in CHO-gmt6 and CHO-gmt7 Cells.

Recombinant MUC1 N-terminal subunit will be purified by a Concanavalin A (Con A) affinity chromatography followed by size-exclusion gel filtration chromatography. The α-mannose residues at the non-reducing end of the Man5GlcNAc2 N-glycan on the recombinant MUC1 subunit will specifically bind Con A linked to the matrix. The MUC1 subunit bound to the column can be eluted by an α-mannoside or an α-glucoside, or by changing pH. The presence of the recombinant MUC1 subunit will be followed by anti-MUC1 antibodies during purification.

### Evaluation of Recombinant MUC1 N-Terminal Subunits as Potential Vaccines in Mice.

Mice will be used as the model animals for testing the efficacy of these recombinant vaccines. B-cell response elicited by the vaccine will be evaluated by measuring the titer of the antibody in immunized mice. The T-cell response will be measured by the activity of CTL in immunized mice.

### Results

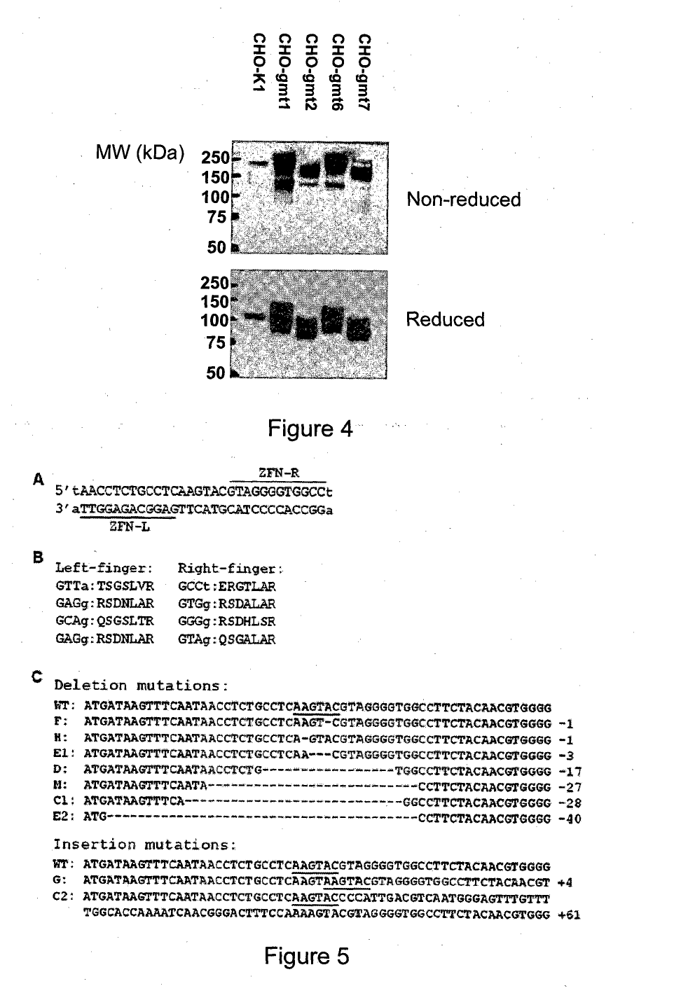

A vector that expresses the N-terminus of MUC1 that contains 5 TRs fused to the Fc of IgG1 (MUC1-Fc) has been generated. Recombinant MUC1-Fc has been produced by wild type CHO-K1 cells and CHO-gmt1, CHO-gmt2, CHO-gmt6 and CHO-gmt7 cells. The results are shown in FIG. 4.

### Example 2

The MUC1 glycopeptide with tumor-associated O-glycans will trigger cancer-specific anti-MUC1 antibody responses. Mannose-terminated N-glycans on the MUC1 will enhance its efficacy by enhancing its uptake by DCs and macrophages.

To investigate the efficacy of recombinant N-terminal subunit of MUC1 as therapeutic vaccines in mouse models and breast cancer patients the following experimental procedure is used:

1. Production of MUC1-Fc fusion proteins.

a. Generation of an expression construct to express MUC1-Fc fusion protein. MUC1 (N terminal subunit) and Fc are linked by a cleavable linker.

b. Production of MUC1-Fc in CHO-gmt5 and CHO-gmt6 cells. For pilot human study, the production will be done by our industry collaborator that has GMP facilities.

2. Mouse Studies

a. Tumor kinetic studies. MUC1 mammary tumors will be established in MUC1 transgenic mice. These mice will be immunized with liposomal preparations of the N-terminal subunit of recombinant MUC1 with empty liposomes as controls.

b. Cytolytic activity. In vaccinated and control mice, CD8 positive cells will be isolated from tumor draining lymph nodes (without any ex-vivo stimulation) and used as effector cells. 51Cr labeled DCs pulsed with MUC1 peptides will be used as target cells. Percentage killing will be measure with 51Cr-release assay.

c. Determination of ADCC. Serum from vaccinated mice will be incubated with 51Cr labelled MUC1 expressing tumor cells. NK cells (effector) will be co-incubated with tumor cells and the release of Cr will be determined.

d. Interferon-gamma Ellispot Assays. CD8+ T cells from the draining lymph nodes of vaccinated mice will be isolated and the level of MUC1 antigen-specific responses will be determined by lnf-g Ellispot Assays.

e. Antibody titres. Levels of anti-MUC1 IgG, IgG1, IgG2, IgG3 and IgM will be determined.

3. Human ex vivo studies

a. DCs cultured and expanded from PBMCs from healthy donors and breast cancer patients will be pulsed with recombinant MUC1 N-terminal subunit produced by CHO-gmt5 and CHO-gmt6 cells, and matured ex-vivo. Autologous mononuclear cells will be co-cultured with these DCs in the presence of cytokines.

b. The phenotype, activation and maturation markers of DCs will be determined by flow cytometry.

c. Supernatant will be analysed for IL-12 and INF-gamma

d. Tumor cell killing. Mononuclear cells from the co-culture will be incubated with Cr 51Cr labelled tumor cells and lytic activity measured. MHC class 1 blocking antibody will be used to determine MHC restriction.

4. Pilot human study. Following animal toxicology studies, a phase I, first-in-man, dose escalation pilot study will be undertaken to explore the safety and tolerability of the vaccine and evaluate the ability of the vaccine to induce MUC1 antigen specific responses in vivo.

### REFERENCES FOR EXAMPLES 1 AND 2

Apostolopoulos V, Pietersz G A, Loveland B E, Sandrin M S, McKenzie I F. (1995) Oxidative/reductive conjugation of mannan to antigen selects for T1 or T2 immune responses. Proc Natl Acad Sci USA. 92(22):10128-32.
Apostolopoulos V, Pietersz G A, Tsibanis A, Tsikkinis A, Drakaki H, Loveland B E, Piddlesden S J, Plebanski M, Pouniotis D S, Alexis M N, McKenzie I F, Vassilaros S. (2006) Pilot phase III immunotherapy study in early-stage breast cancer patients using oxidized mannan-MUC1 [ISRCTN71711835]. Breast Cancer Res. 8(3):R27.
Bafna S, Kaur S, Batra S K. (2010) Membrane-bound mucins: the mechanistic basis for alterations in the growth and survival of cancer cells. Oncogene. 29(20):2893-904.
Bäckström M, Link T, Olson F J, Karlsson H, Graham R, Picco G, Burchell J, Taylor-Papadimitriou J, Noll T, Hansson G C. (2003) Recombinant MUC1 mucin with a breast cancer-like O-glycosylation produced in large amounts in Chinese-hamster ovary. Biochem J. 376:677-86.
Beatson R E, Taylor-Papadimitriou J, Burchell J M. (2010) MUC1 immunotherapy. Immunotherapy. 2(3):305-27.
Blixt O, Bueti D, Burford B, Allen D, Julien S, Hollingsworth M, Gammerman A, Fentiman I, Taylor-Papadimitriou J, Burchell J M. (2011) Autoantibodies to aberrantly glycosylated MUC1 in early stage breast cancer are associated with a better prognosis. Breast Cancer Res. 13(2):R25.
Brockhausen I. (2006) Mucin-type O-glycans in human colon and breast cancer: glycodynamics and functions. EMBO Rep. 7(6):599-604.
Ding L, Lalani E N, Reddish M, Koganty R, Wong T, Samuel J, Yacyshyn M B, Meikle A, Fung P Y, Taylor-Papadimitriou J, et al. (1993) Immunogenicity of synthetic peptides related to the core peptide sequence encoded by the human MUC1 mucin gene: effect of immunization on the growth of murine mammary adenocarcinoma cells transfected with the human MUC1 gene. Cancer Immunol Immunother. 36(1):9-17.
Dube D H, Bertozzi C R. (2005) Glycans in cancer and inflammation—potential for therapeutics and diagnostics. Nat Rev Drug Discov. 4(6):477-88.
Finke L H, Wentworth K, Blumenstein B, Rudolph N S, Levitsky H, Hoos A. (2007) Lessons from randomized phase Ill studies with active cancer immunotherapies—outcomes from the 2006 meeting of the Cancer Vaccine Consortium (CVC). Vaccine. 27; 25 Suppl 2:B97-B109.
Freire T, Zhang X, Dériaud E, Ganneau C, Vichier-Guerre S, Azria E, Launay O, Lo-Man R, Bay S, Leclerc C. (2010) Glycosidic Tn-based vaccines targeting dermal dendritic cells favor germinal center B-cell development and potent antibody response in the absence of adjuvant. Blood. 116(18):3526-36.
Friedman B, Vaddi K, Preston C, Mahon E, Cataldo J R, McPherson J M. (1999) A comparison of the pharmacological properties of carbohydrate remodeled recombinant and placental-derived beta-glucocerebrosidase: implications for clinical efficacy in treatment of Gaucher disease. Blood. 93(9):2807-16.
Gazi U, Martinez-Pomares L. (2009) Influence of the mannose receptor in host immune responses. Immunobiology. 214(7):554-61.
Gilewski T A, Ragupathi G, Dickler M, Powell S, Bhuta S, Panageas K, Koganty R R, Chin-Eng J, Hudis C, Norton L, Houghton A N, Livingston P O. (2007) Immunization of high-risk breast cancer patients with clustered sTn-KLH conjugate plus the immunologic adjuvant QS-21. Clin Cancer Res. 13(10):2977-85.
Goh J, Zhang P, Chan K F, Lee M M, Lim S F, and Song Z. (2010) RCA-I-resistant CHO mutant cells contain dysfunctional GnT-I and expression of GnT I in these mutants enhances sialylation of recombinant erythropoietin. Metab. Eng. 12(4):360-368.
Goldman B., DeFrancesco L. (2009) The cancer vaccine roller coaster. Nat. Biotechnol. 27(2):129-39.
Hanisch F G, Schwientek T, Von Bergwelt-Baildon M S, Schultze J L, Finn O. (2003) O-Linked glycans control glycoprotein processing by antigen-presenting cells: a biochemical approach to the molecular aspects of MUC1 processing by dendritic cells. Eur J Immunol. 33(12):3242-54.
Hattrup C L, Gendler S J. (2008) Structure and function of the cell surface (tethered) mucins. Annu Rev Physiol. 70:431-57.
Hokke C H, Bergwerff A A, Van Dedem G W, Kamerling J P, Vliegenthart J F. (1995) Structural analysis of the sialylated N- and O-linked carbohydrate chains of recombinant human erythropoietin expressed in Chinese hamster ovary cells. Eur J Biochem. 228(3):981-1008.
Hollingsworth M A, Swanson B J. (2004) Mucins in cancer: protection and control of the cell surface. Nat Rev Cancer. 4(1):45-60.
Holmberg L A, Oparin D V, Gooley T, Lilleby K, Bensinger W, Reddish M A, MacLean G D, Longenecker B M, Sandmaier B M. (2000) Clinical outcome of breast and ovarian cancer patients treated with high-dose chemotherapy, autologous stem cell rescue and THERATOPE STn-KLH cancer vaccine. Bone Marrow Transplant. 25(12):1233-41
Hull S R, Bright A, Carraway K L, Abe M, Hayes D F, Kufe D W. (1989) Oligosaccharide differences in the DF3 sialomucin antigen from normal human milk and the BT-20 human breast carcinoma cell line. Cancer Commun. 1(4):261-7.
Julien S, Picco G, Sewell R, Vercoutter-Edouart A S, Tarp M, Miles D, Clausen H, Taylor-Papadimitriou J, Burchell J M. (2009) Sialyl-Tn vaccine induces antibody-mediated tumour protection in a relevant murine model. Br J Cancer. 100(11):1746-54.
Julien S, Delannoy P. (2003) Sialyl-Tn antigen in cancer: from diagnosis to therapy Recent Research Developments in Cancer Kerala: Transworld Reserach Network; 185-199. Pandalai S G (ed) Vol. 5, pp.
Keppler-Ross S, Douglas L, Konopka J B, Dean N. (2010) Recognition of yeast by murine macrophages requires mannan but not glucan. Eukaryot Cell. 9(11):1776-87.
Kufe D W. (2009) Mucins in cancer: function, prognosis and therapy. Nat Rev Cancer. 9(12):874-85.
Lim S F, Lee M M, Zhang P and Song Z. (2008) The Golgi CMP-sialic acid transporter: A new CHO mutant provides functional insights. Glycobiology 18(11):851-60.
Link T, Bäckström M, Graham R, Essers R, Zörner K, Gätgens J, Burchell J, Taylor-Papadimitriou J, Hansson G C, Noll T. (2004) Bioprocess development for the production of a recombinant MUC1 fusion protein expressed by CHO-K1 cells in protein-free medium. J Biotechnol. 110(1):51-62.
Lloyd K O, Burchell J, Kudryashov V, Yin B W, Taylor-Papadimitriou J. (1996) Comparison of O-linked carbohydrate chains in MUC-1 mucin from normal breast epithelial cell lines and breast carcinoma cell lines. Demonstration of simpler and fewer glycan chains in tumor cells. J Biol Chem. 271(52):33325-34.
Loveland B E, Zhao A, White S, Gan H, Hamilton K, Xing P X, Pietersz G A, Apostolopoulos V, Vaughan H, Karanikas V, Kyriakou P, McKenzie I F, Mitchell P L. (2006) Mannan-MUC1-pulsed dendritic cell immunotherapy: a phase I trial in patients with adenocarcinoma. Clin Cancer Res. 12(3 Pt 1):869-77.
McGreal E P, Miller J L, Gordon S. (2005) Ligand recognition by antigen-presenting cell C-type lectin receptors. Curr Opin Immunol. 17(1):18-24.
Miles D, Papazisis K. (2003) Rationale for the clinical development of STn-KLH (Theratope) and anti-MUC-1 vaccines in breast cancer. Clin Breast Cancer. 3 (Suppl 4):S134-S138.
North S J, Huang H H, Sundaram S, Jang-Lee J, Etienne A T, Trollope A, Chalabi S, Dell A, Stanley P, Haslam S M. (2010) Glycomics profiling of Chinese hamster ovary cell glycosylation mutants reveals N-glycans of a novel size and complexity. J Biol Chem. 285(8):5759-75.
Olson F J, Backstrom M, Karlsson H, Burchell J, Hansson G C. (2005) A MUC1 tandem repeat reporter protein produced in CHO-K1 cells has sialylated core 1 O-glycans and becomes more densely glycosylated if coexpressed with polypeptide-GalNAc-T4 transferase. Glycobiology. 15(2):177-91.
Patnaik S K, Stanley P. (2006) Lectin-resistant CHO glycosylation mutants. Methods Enzymol. 416:159-82.
Potapenko I O, Haakensen V D, Lüders T, Helland A, Bukholm I, Sørlie T, Kristensen V N, Lingjaerde O C, Børresen-Dale A L. (2010) Glycan gene expression signatures in normal and malignant breast tissue; possible role in diagnosis and progression. Mol Oncol. 4(2):98-118.
Plebanski M, Katsara M, Sheng K C, Xiang S D, Apostolopoulos V. (2010) Methods to measure T-cell responses. Expert Rev Vaccines. 9(6):595-600.
Rachagani S, Torres M P, Moniaux N, Batra S K. (2009) Current status of mucins in the diagnosis and therapy of cancer. Biofactors. 35(6):509-27.
Ragupathi G, Howard L, Cappello S, Koganty R R, Qiu D, Longenecker B M, Reddish M A, Lloyd K O, Livingston P O. (1999) Vaccines prepared with sialyl-Tn and sialyl-Tn trimers using the 4-(4-maleimidomethyl)cyclohexane-1-carboxyl hydrazide linker group result in optimal antibody titers against ovine submaxillary mucin and sialyl-Tn-positive tumor cells. Cancer Immunol Immunother. 48(1):1-8.
Richardson J P, Macmillan D. (2008) Mucin-Based Vaccines. Glycoscience. Vol 2, Part 12, p 2645-2698, DOI: 10.1007/978-3-540-30429-668
Rughetti A, Pellicciotta I, Biffoni M, Backstrom M, Link T, Bennet E P, Clausen H, Noll T, Hansson G C, Burchell J M, Frati L, Taylor-Papadimitriou J, Nuti M. (2005) Recombinant tumor-associated MUC1 glycoprotein impairs the differentiation and function of dendritic cells. J Immunol. 174(12):7764-72.
Sørensen A L, Reis C A, Tarp M A, Mandel U, Ramachandran K, Sankaranarayanan V, Schwientek T, Graham R, Taylor-Papadimitriou J, Hollingsworth M A, Burchell J, Clausen H. (2006) Chemoenzymatically synthesized multimeric Tn/STn MUC1 glycopeptides elicit cancer-specific anti-MUC1 antibody responses and override tolerance. Glycobiology. 16(2):96-107.
Soares M M, Mehta V, Finn O J. (2001) Three different vaccines based on the 140-amino acid MUC1 peptide with seven tandemly repeated tumor-specific epitopes elicit distinct immune effector mechanisms in wild-type versus MUC1-transgenic mice with different potential for tumor rejection. J Immunol. 166(11):6555-63.
Springer G F. (1984) T and Tn, general carcinoma autoantigens. Science. 224(4654):1198-206.
Springer G F. (1997) Immunoreactive T and Tn epitopes in cancer diagnosis, prognosis, and immunotherapy. J Mol Med. 75(8):594-602.
Tang C K, Katsara M, Apostolopoulos V. (2008) Strategies used for MUC1 immunotherapy: human clinical studies. Expert Rev Vaccines. 7(7):963-75.
Tarp M A, Clausen H. (2008) Mucin-type O-glycosylation and its potential use in drug and vaccine development. Biochim Biophys Acta. 1780(3):546-63.
Tarp M A, Sørensen A L, Mandel U, Paulsen H, Burchell J, Taylor-Papadimitriou J, Clausen H. (2007) Identification of a novel cancer-specific immunodominant glycopeptide epitope in the MUC1 tandem repeat. Glycobiology. 2007 February; 17(2):197-209.
Taylor-Papadimitriou J, Burchell J, Miles D W, Dalziel M. (1999) MUC1 and cancer Biochim Biophys Acta. 1455(2-3):301-13.
Thornton D J, Rousseau K, McGuckin M A. (2008) Structure and function of the polymeric mucins in airways mucus. Annu Rev Physiol. 70:459-86.
Van Patten S M, Hughes H, Huff M R, Piepenhagen P A, Waire J, Qiu H, Ganesa C, Reczek D, Ward P V, Kutzko J P, Edmunds T. (2007) Effect of mannose chain length on targeting of glucocerebrosidase for enzyme replacement therapy of Gaucher disease. Glycobiology. 17(5):467-78.
Vlad A M, Finn O J. (2004) Glycoprotein tumor antigens for immunotherapy of breast cancer. Breast Dis. 20:73-9.
Vlad A M, Muller S, Cudic M, Paulsen H, Otvos L Jr, Hanisch F G, Finn O J. (2002) Complex carbohydrates are not removed during processing of glycoproteins by dendritic cells: processing of tumor antigen MUC1 glycopeptides for presentation to major histocompatibility complex class II-restricted T cells. J Exp Med. 196(11):1435-46.
von Mensdorff-Pouilly S, Petrakou E, Kenemans P, van Uffelen K, Verstraeten A A, Snijdewint F G, van Kamp G J, Schol D J, Reis C A, Price M R, Livingston P O, Hilgers J. (2000) Reactivity of natural and induced human antibodies to MUC1 mucin with MUC1 peptides and n-acetylgalactosamine (GalNAc) peptides. Int J Cancer. 86(5):702-12.
Wandall H H, Blixt O, Tarp M A, Pedersen J W, Bennett E P, Mandel U, Ragupathi G, Livingston P O, Hollingsworth M A, Taylor-Papadimitriou J, Burchell J, Clausen H. (2010) Cancer biomarkers defined by autoantibody signatures to aberrant O-glycopeptide epitopes. Cancer Res. 70(4):1306-13.
Xing P X, Apostolopoulos V, Pietersz G, McKenzie I F. (2001) Anti-mucin monoclonal antibodies. Front Biosci. 6:D1284-95.
Zhang S, Graeber L A, Helling F, Ragupathi G, Adluri S, Lloyd K O, Livingston P O. (1996) Augmenting the immunogenicity of synthetic MUC1 peptide vaccines in mice. Cancer Res. 56(14):3315-9.

### Example 3

### GDP-Fucose Transporter Mutant

It is now widely recognized that removal of the fucose from the N-glycans attached to Asn297 of human IgG1 significantly enhances its binding to its receptor, FcγIIIa, and thereby dramatically improves antibody-dependent cellular cytotoxicity (ADCC) (12;13). Recent reports have shown that removal of sialic acid from IgG1 also enhanced ADCC in vivo (14;15). As disclosed above, we have generated a CHO mutant line that has a dysfunctional CMP-sialic acid transporter (16). As a result, glycoproteins produced by this cell line are completely free of sialic acid. The mutant line was originally named MAR-11 and has since been renamed as CHO-gmt1, for CHO glycosylation mutant-1. We utilized the zinc finger nuclease technology and successfully knocked out the gene encoding GFT from the CHO-gmt1 cells. The resultant cell line, CHOgmt5, was shown to produce N-glycans essentially free of sialic acid and fucose. We ultimately aim to test whether an IgG1 lacking both fucose and sialic acid would perform even better in activating ADCC. However, in this study we have focused our efforts on making use of this mutant cell line to study the structure-function relationship of GFT. The activity of GFT was indirectly reflected by the binding of a fucose-specific lectin, Aleuria Aurantia lectin (AAL), to the CHO-gmt5 cells. Using this system, we successfully identified elements located in different regions of GFT with significant impacts on its transport activity.

### Experimental Procedures

### Materials

Monoclonal anti-hemagglutinin (HA) antibody (Clone HA-7), rabbit polyclonal anti-B4GalTI antibodies were purchased from Sigma (St. Louis, Mo.). Rabbit polyclonal antibodies for giantin, GM130, mannosidase II (ManII), and TGN46 were purchased from Abcam (Cambridge, UK). Biotinylated AAL was purchased from Vector Laboratories (Burlingame, Calif.). FITC-conjugated PNA was purchased from EY Laboratories (San Mateo, Calif.). AlexaFluor dye-conjugated secondary probes, including goat anti-mouse IgG, goat anti-rabbit IgG, and streptavidin, were purchased from Invitrogen (Eugene, Oreg.). Cy3-conjugated streptavidin were purchased from Jackson ImmunoResearch (West Grove, Pa.). Trypsin was purchased from Promega (Madison, Wis.). NGlycosidase F (PNGase F) was purchased from Calbiochem (San Diego, Calif.). Hypercarb SPE cartridges (200 mg sorbent bed weight) were from ThermoFisher Scientific (Waltham, Mass.). 2, 5-Dihydroxybenzoic acid (DHB) and Sep-Pak Vac C18 cartridge were from Waters Corporation (Milford, Mass.). Acetic acid, ammonium bicarbonate, hydrochloric acid, methy iodide, sodium acetate and sodium hydroxide (NaOH) were all of analytical reagent grade from Merck (KGaA, Germany). Acetonitrile, dimethyl sulfoxide and methanol were of HPLC grade from Merck. Chloroform was of HPLC grade from Fisher Scientific (Pittsburgh, Pa.). Ultrapure water (Sartorius, Goettingen, Germany) was used throughout the analysis.

### Cell Culture and Transfection of the ZFN Constructs—

CHO-gmt1 cells, formerly called MAR-11 (16), were cultured in Dulbecco's Modified Eagle's Medium supplemented with 10% fetal bovine serum (FBS) (both from Invitrogen, Auckland, Calif.), at 37° C. with 5% CO₂. Prior to transfection, 5×10⁵ cells were seeded into each well of 6-well plates and cultured overnight. The constructs for expressing the pair of ZFNs were transiently transfected into CHO-gmt1 cells using Lipofectamine 2000 (Invitrogen, USA) according to manufacturer's protocol. In each transfection, a total of 4 μg plasmid DNA (2 μg for each ZFN) and 10 μl Lipofectamine 2000 reagent in 500 μl serum-free DMEM were added to each well containing 2 ml of medium. After overnight incubation, the transfection reagent was replaced with normal culture medium and cultured for 2 days before single cell cloning into 96-cell plates.

### Designing the ZFNs to Target the GDPfucose Transporter in CHO Cells—

The “modular assembly” method was used to generate specific ZFNs for targeting the GFT gene in CHO cells. The open reading frame of the Chinese hamster GDP-fucose transporter was analyzed using the web-based ZiFiT program provided by the Zinc Finger Consortium (ZiFiT: software for engineering zinc finger proteins (V3.0)) at: http://bindr.gdcb.iastate.edu/ZiFiT/ (17). The ZiFiT output located a DNA sequence in the first exon of the GDP-fucose transporter coding region (5′-tAACCTCTGCCTCAAGTACGTAGGGGTGG CCt-3′) (SEQ ID NO: 14) as a potential target site for ZFNs (FIG. 5A). Two binding sites for ZFNs (underlined) are separated by 6 bps which is the optimal distance for cleavage by two ZFNs. This sequence perfectly matches the ideal target sequence for 4-fingered ZFNs which is:

(SEQ ID NO: 42)
5′-NNCNNCNNCNNCXXXXXXGNNGNNGNN GNN-3′.

It allows each zinc finger in the left ZFN and the right ZFN to bind to a 5′-GNN-3′ DNA triplet (FIG. 5A). The fingers that bind the 5′-GNN-3′ triplets are the best studied and strong DNA-binding fingers (18-20). The structural scaffold for the ZFNs was adopted from previous publications (21;22). To eliminate unwanted homodimerization of Fokl cleavage domain, the high-fidelity Fokl-KK and Fokl-EL variants were used (23). Each zinc finger in our ZFNs was designed based on the publicly available information described in the literature. The complete design of the left and right finger is shown in FIG. 5B.

Isolation and Characterization of CHO-gmt5 Cells, CHO-gmt1 Cells with Inactivated GDPfucose Transporter Gene—

Two days after transfection single cells were seeded in 96-well plates for clone isolation. Genomic DNA from each single clone was isolated and the GDPfucose transporter target locus was amplified by PCR. The sequence of the forward PCR primer is 5′-GGCGCCTCTGAAGCGGTCCAGGATCC (SEQ ID NO: 43). The sequence for the reverse PCR primer is 5′-GCCACATGTGAGCAGGGCATAGAAGG (SEQ ID NO: 44). A 520-bp PCR product was generated from the wild type CHO genomic sequence which can be digested by a restriction enzyme SnaBI (TACIGTA) around the ZFN restriction site (FIG. 5A) to give rise to a smaller fragment of 114-bp and a larger fragment of 406-bp. In case a mutation has occurred at the targeted site by the ZFNs, the PCR product may become resistant to the restriction enzyme. However, for insertion mutants, the PCR products have to be sequenced in order to identify the mutations.

Analysis of the Oligosaccharides Released from Recombinant EPO-FC Using Matrix Assisted Laser Desorption/Ionization Time-of Flight Mass Spectrometry (MALDI-TOF)—

The recombinant EPO-Fc was produced in CHOgmt1 cells and CHO-gmt5 cells as described earlier (24) for N-glycan structure analyses. Briefly, the Fc region from human IgG1 was fused to the C-terminus of EPO by overlap PCR. The PCR product that encodes the EPO-Fc fusion was cloned into pcDNA3.1 with a Kozak sequence placed upstream of the translation start codon ATG. The EPO-Fc construct was transiently transfected into CHO-gmt1 and CHO-gmt5 cells. Recombinant EPO-Fc produced was purified with a Protein A column. An amount of 100 μg of purified EPO-Fc was used for carbohydrate structure analysis. The carbohydrates liberated from the EPO-Fc by PNGase F were analyzed by matrix assisted laser desorption/ionization time-of-flight mass spectrometry (MALDI-TOF) analysis as described previously (16).

### Immunofluorescence Microscopy—

CHOgmt5 cells were plated on glass coverslips in 6-well plates and transfected next day using FuGene 6 reagent (Roche, Indianapolis, Ind.) with plasmid constructs for different GFT variants. Two days after transfection, cells were washed with PBS, fixed with 4% paraformaldehyde (PFA) (diluted from 16% methanol-free PFA from Thermo Scientific (Rockford, Ill.)) in PBS for 10 min and permeabilized by 0.1% Triton X-100 (Sigma, St. Louis, Mo.) in PBS for 5 min. After washing with PBS twice, cells were blocked with Carbo-Free blocking solution (Vector Laboratories, Burlingame, Calif.), briefly rinsed with PBS and then incubated with PBS containing 1% BSA and a monoclonal anti-HA antibody (5 μg/ml final concentration) as well as a Golgi marker antibody at an appropriate dilution for 1 hour. For experiments involving the analysis of GFT activity, biotinylated AAL (5 μg/ml final concentration) was included in the primary antibody mixture. The coverslips were then washed three times in PBS and incubated with secondary antibodies, i.e. AlexaFluor 488 conjugated goat anti-mouse IgG (for the detection of HA-GFT) and AlexaFluor 594 conjugated goat anti-rabbit IgG (for the detection of Golgi marker), as well as AlexaFluor 647 conjugated-streptavidin for experiments involving biotinylated AAL. In all experiments, Hoechst 33342 (Invitrogen, Eugene, Oreg.) at 2 μg/ml (final concentration) was included in the secondary antibody mixture to stain the nuclei. Finally, the coverslips were washed four times in PBS and then mounted onto glass slides using ProlongGold antifade medium (Invitrogen, Eugene, Oreg.). Fluorescence images were taken using a Carl Zeiss LSM 510 META confocal microscope with a 63×PLANapochromat objective (1.40 NA) immersed in oil. All AAL images within the same set of experiments were acquired under constant imaging parameters. Representative areas were cropped with LSM Image Brower and used to produce the final figures. For localization analysis of GFT, images were taken at 10 successive optical slices in the Z-direction (0.41 μm thickness per slice) and used to produce the final figure.

### FACS Analysis of Cell Surface Fucosylation

CHO-gmt5 cells were transfected with different GFT constructs in 12-well plates using the method described above. Two days after transfection, cells were washed with PBS and harvested using PBS containing 2 mM EDTA. After washing with PBS, cells were fixed with 4% PFA (diluted from 16% methanol-free PFA. Thermo Scientific) in PBS for 10 min. After washing with PBS twice, cells were incubated with Carbo-Free blocking solution for 30 min, and then incubated with PBS containing 1% BSA and 5 μg/ml biotinylated AAL for 30 min. After 2 washes with PBS, cells were incubated in PBS containing 1% BSA and 5 μg/ml Cy3-conjugated streptavidin for 30 min. Finally, cells were washed twice in PBS and analyzed by a BD LSR II FACS analyzer. For each run, 10,000 events were recorded. AAL-positive region was gated based on untransfected CHO-gmt5 cells and cells transfected with a full-length GFT construct. Under such gating parameters, CHOgmt5 cells transfected with a T306R construct had <0.5% population in the AAL-positive region. To control for transfection efficiency, a separate set of transfections were done with each GFT construct mixed with a GFP construct at 10:1 ratio. Cells were stained by the same method described above and GFP fluorescence was analyzed by FACS. Similar GFP fluorescence profiles were obtained for all transfections, suggesting comparable transfection efficiencies for all constructs.

### Results

ZFNs generated by the combinatorial selection-based methods may have high DNAbinding affinity and low toxicity. However, it requires large randomized libraries and the selection expertise that only a few labs possess. We engineered a pair of ZFNs to target the GDP-fucose gene in CHO cells using a simplified “modular assembly” strategy. Each zinc finger in our ZFNs was designed based on the publicly available information described in the literature. We have demonstrated that this method can be a viable approach for researchers who do not have the expertise to perform a combinatorial selection.

### Design of ZFNs to Target the GDP-Fucose Transporter in CHO Cells—

The open reading frame of the Chinese hamster GDP-fucose transporter was analyzed using the web-based ZiFiT program provided by the Zinc Finger Consortium (ZiFiT: software for engineering zinc finger proteins (V3.0)) at: http://bindr.gdcb.iastate.edu/ZiFiT/ (17;25). One potential target site in the first exon of the GDPfucose transporter coding region (5′-tAACCTCTGCCTCAAGTACGTAGGGGTGG CCt-3′) (SEQ ID NO: 14) was identified (FIG. 5A). Two potential binding sites for ZFNs (underlined) are separated by 6 bps which is the optimal distance for cleavage by two ZFNs. This sequence perfectly matches the ideal target sequence for 4-fingered ZFNs which is: 5′-NNCNNCNNCNNCXXXXXXGNNGNNGNN GNN-3′ (SEQ ID NO: 42). It allows each zinc finger motif in the left ZFN and the right ZFN to bind to a 5′-GNN-3′ DNA triplets (FIG. 5A) as it has been shown that the 5′-GNN-3′ triplets generally show high affinities for proper zinc fingers (18-20). The structural scaffold for the ZFNs was adopted from previous publications (21;22). To eliminate unwanted homodimerization of Fokl cleavage domain, the high-fidelity Fokl-KK and Fokl-EL variants were used (23). The DNA-binding domains of the two ZFNs against this site were assembled using an archive of engineered zincfinger motifs collected from previous publications, mainly by Barbas's group and Sangamo BioSciences Inc. The complete design of the left and right finger is shown in FIG. 5B.

### Generation and Identification of GDPfucose Transporter-Deficient CHO Cells—

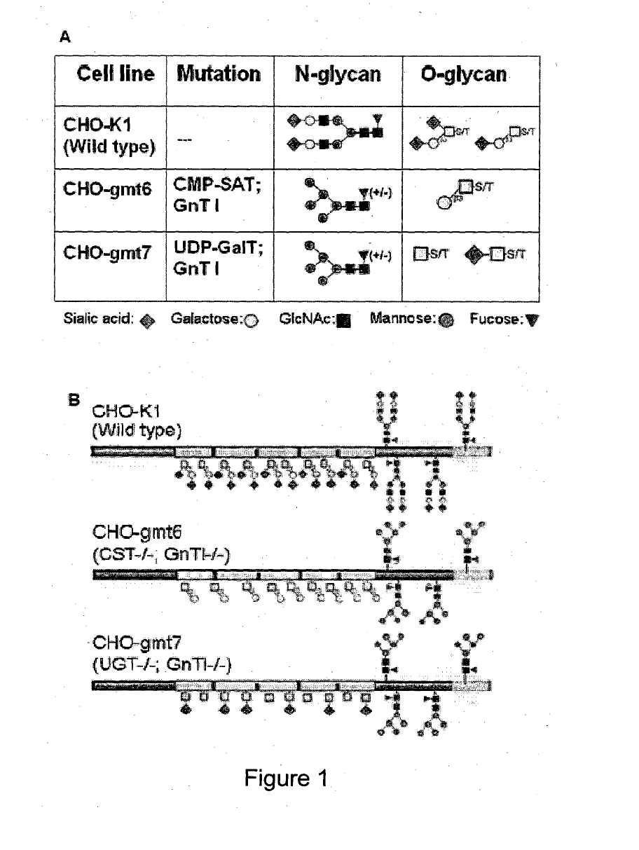

CHOgmt1 cells were transfected with the vectors that express the ZFNs to bind the ZFN-L and ZFN-R shown in FIG. 5A. Two days after transfection single cells were seeded in 96-well plates for clone isolation. Genomic DNA from each single clone was isolated and the GDP-fucose transporter target locus was amplified by specific PCR primers. A 520-bp PCR product was generated from the wild-type CHO genomic sequence which can be digested by a restriction enzyme SnaBI (TACIGTA) around the ZFN restriction site (FIG. 5A) to give rise to a smaller fragment of 114-bp and a larger fragment of 406-bp. In the event that a deletion mutation has occurred at the targeted site by the ZFNs, the PCR product may become resistant to the restriction enzyme. However, for insertion mutants, the PCR products have to be sequenced in order to identify the mutations. DNA sequencing results for the PCR products of the isolated clones showed several random deletion and insertion mutations around the ZFN target site (FIG. 1C). C, D, E, F, G and M represent different single clones in which mutations have been identified. While clone D, F, G and M each has one mutated allele, they all still possessed one wild-type allele of the GFT gene. As a result, these cells still express fucose-containing glycoproteins as suggested by the positive staining of a fucose-specific lectin, AAL (data not shown). Two alleles of the GFT gene in the clone C were mutated, resulting in one deletion mutation (C1) and one insertion mutation (C2). Unfortunately, this clone had three alleles of the GFT gene and the third was not mutated. Therefore, clone C also possessed a wild type phenotype. Interestingly, the inserted DNA fragment shown in C2 came from the expression vector used in this experiment. Clone E had two alleles of the GFT gene and both were mutated, each with a deletion mutation. E1 has a 3-bp deletion, resulting in mutation of K137-Y138 to one N residue. E2 has a 40-bp deletion which resulted in a prematurely truncated GFT product of 172 amino acids. The PCR product of this clone was found to be resistant to the restriction enzyme SnaBI because the recognition site (TACIGTA) in both alleles had been abolished by the deletions. This clone, named CHO-gmt5, was used for further fucosylation analyses.

### Endogenous and Recombinant N-Glycans Produced by CHO-gmt5 Cell are Free of Fucose—

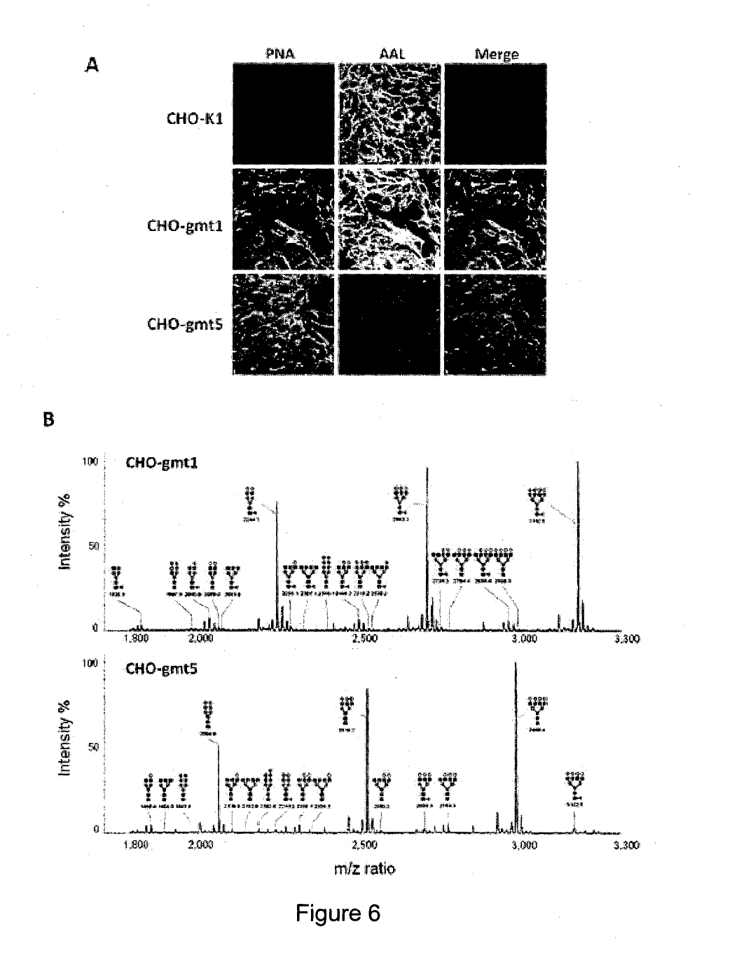

Lectin staining was performed to gain insights into the glycan terminal structures on CHOgmt5 cells (FIG. 6A). Parental and wild-type cell lines, CHO-gmt1 and CHO-K1, were also included in this analysis. Cells were labeled with two lectins, PNA which recognizes terminal galactose residue, and AAL which recognizes fucose residue. CHO-K1 is negative for PNA binding due to the capping of galactose residues by sialic acids. It is positive for AAL binding because of the abundant fucosylation of Nglycans. In contrast, CHO-gmt1 is positive not only for AAL, but also for PNA due to the genetic defect in CST leading to production of galactose-terminated N-glycans. As expected, CHO-gmt5 was shown to be positive for PNA. However, it is negative for AAL binding, suggesting the lack of fucosylation due to knockout of the GFT gene (note the weak staining of CHO-gmt5 by AAL is the background, which is consistent with a previous publication) (26). Recombinant EPO-Fc fusion protein produced in CHO-gmt1 (MAR-11) and CHOgmt5 were purified and the N-linked glycans liberated by PNGase F were analyzed by MALDI-TOF as previously described (16) and the mass spectra are as shown in FIG. 6B. The glycans isolated from CHO-gmt1 were mostly fucosylated, and the dominant species were the asialo, core-fucosylated galactosyl biantennary, triantennary and tetraantennary glycans. In contrast, the dominant species isolated from CHO-gmt5 were the asialo, afucosylated galactosyl biantennary, triantennary and tetraantennary glycans, correspondingly. Minor peaks corresponding to fucosylated species were observed in CHO-gmt5 sample at residual levels, but these peaks were also control in a control sample where CHO-gmt5 cells were not even transfected with an EPO-Fc construct (data not shown), suggesting these residual amounts of fucosylated glycans might come from the culture medium. These data clearly demonstrated that glycoproteins expressed on the cell surface of, or secreted by, CHO-gmt5 cells are completely free of fucose due to a dysfunctional GTF gene.

### The Human GDP-Fucose Transporter is Localized to Medial- and Trans-Golgi—

The C. elegans and human GFT have been shown to be localized to the Golgi apparatus (9).

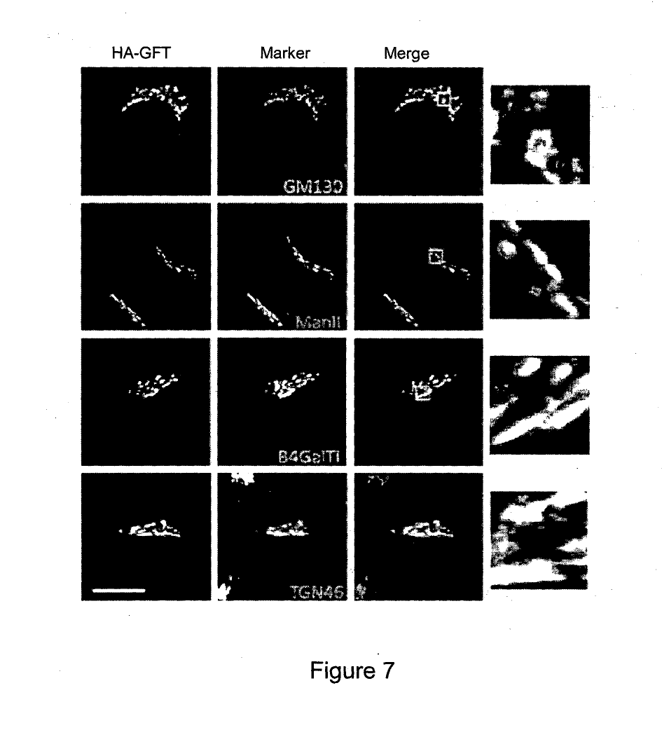

To ascertain the distribution of GFT in the Golgi, we transiently expressed HA-tagged GFT in HeLa cells and analyzed the distribution of GFT by immunofluorescence microscopy and compared its localization pattern with defined Golgi cisternal markers. Cells expressing moderate level of GFT were analyzed and representative images were shown in FIG. 7. GFT did not show co-localization with cis-Golgi marker GM130, or with TGN marker TGN46. Partial colocalization was detected with medial-Golgi marker ManII and high degree of co-localization was seen with trans-Golgi marker B4GalTI. This indicates that at steady state, the majority of GFT protein is localized to the trans-Golgi with a smaller fraction localized to medial-Golgi. This observation is consistent with the distribution pattern of CST in HeLa cells (27).

### Discussion

Recently, another Slc35 protein, ER localized Efr in Drosophila (30) and ERGIC/cis-Golgi-localized mammalian Slc35c2 (31), was shown to have transport activity for GDP-fucose and regulate the O-fucosylation of Notch. Overexpression of Slc35c2 was shown to compete with the canonical GFT and have an inhibitory effect on the formation of Lewis-X structure but imposes a marginal effect on core fucosylation of N-glycans (31). We analyzed the localization of GFT in HeLa and found it to have more co-localization with trans-Golgi marker and to a lesser extent with medial-Golgi marker (FIG. 7). Therefore, the spatial segregation of Slc35c2 and GFT explains why Slc35c2 regulates fucosylation of Notch, which takes place pre-Golgi, and does not affect core fucosylation of N-glycans. Thus, targeting GFT is necessary and sufficient for regulating core fucosylation of N-glycans at membrane transport level.

Absence of core fucose on Fc N-glycan has been shown to enhance the ADCC effect of IgG1 (12;13). CHO GFT−/− cell line has been generated by homologous recombination and used as a host cell line for the production of fucose-free recombinant antibody (32). Absence of sialic acid on Fc N-glycan is also favorable for ADCC effect (14;15). It is tempting to speculate that the absence of both fucose and sialic acid will offer combinatorial enhancement to the ADCC effect. Our CHO-gmt5 with double genetic defects in GFT and CST is capable of producing recombinant glycoproteins free of fucose and sialic acid and thus, has the potential application for the production of recombinant antibodies with improved ADCC effect. In fact, our ongoing study has demonstrated that CHOgmt mutants (including CHO-gmt1 and 5) can be adapted to suspension culture in protein-free media with growth profiles similar to that of wild-type CHO-K1 cells.

CHO glycosylation mutants with genetic defects in multiple NSTs (such as CST and UGT) have been isolated and reported (7). Our CHOgmt5 is a new addition to this range of mutants and holds promise for further targeting of additional NSTs towards a mammalian cell line with minimal NST background for the analysis of NST activity and specificity. The abbreviations used are: AAL, Aleuria Aurantia lectin; CST, CMP-sialic acid transporter; FACS, fluorescence-activated cell sorting; GFT, GDP-fucose transporter; GMT, GDP-mannose transporter; NST, nucleotide-sugar transporter; PNA, peanut agglutinin; UGT, UDP-galactose transporter; ZFN, zinc-finger nuclease.

### Example 4

### Generating CHO-gmt3 Cells by Inactivating GDP-Fucose Transporter in CHO-K1 Cells with Zinc-Finger Nucleases

Wild type CHO-K1 cells were first seeded overnight into 6-well plates and transfected with plasmid DNA encoding for the left and right zinc-finger nucleases (ZFNs) targeting the GDP-fucose transporter as described by Zhang et al. (2012), and above. Transfected cells were then grown to near confluence under non-selective conditions and subcultured for 2 passages.

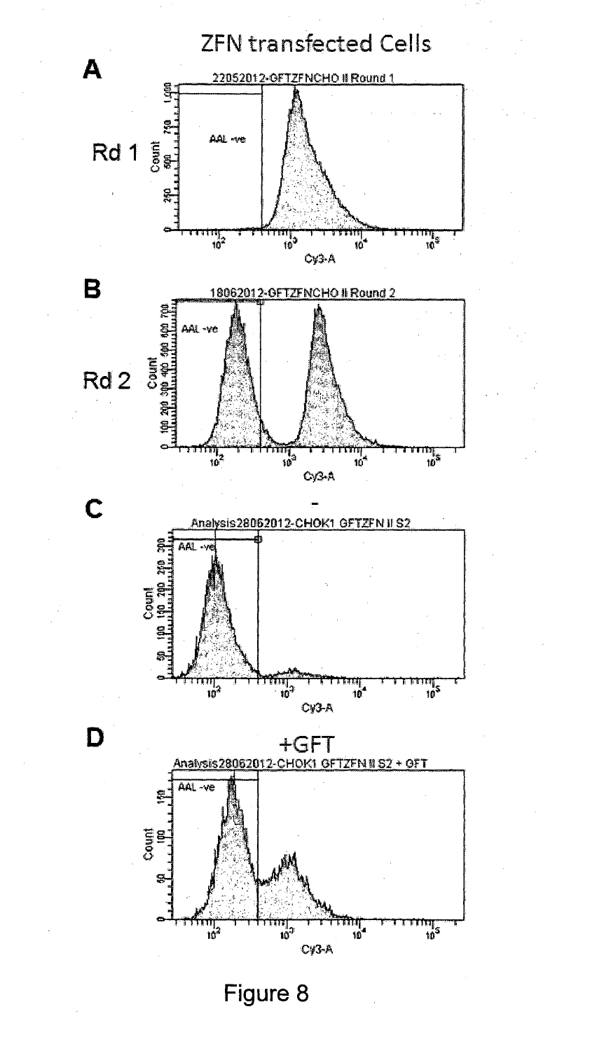

Cells with GDP-fucose transporter successfully inactivated by the ZFNs were isolated by FACS using a fucose-specific lectin, AAL (Aleuria aurantia lectin). To prepare for FACS sorting, 10 million ZFN transfected cells were incubated with biotinylated AAL lectin and stained with Cy3-conjugated streptavidin. Stained cells were sorted on a 5-laser Becton Dickenson FACSAria Ilu SORP cell sorter. As shown in FIG. 8, the sorting gate for AAL-negative (AAL-ve) cells was set to collect the lowest 0.5% of AAL-stained cells. About 12000 cells were collected out of 3.5 million cells processed through the sorter (FIG. 8A). These cells were cultured and grown for ˜2 weeks before subjecting to a second round of sorting. Second round of FACS showed that more than 40% of the cells are AAL-ve after the first round of FACS enrichment (FIG. 8B). About 1 million AAL-ve cells were collected out of 4 million cells sorted this time.

Further FACS analysis of the resultant enriched pool showed that most of the cells were AAL-ve cells (FIG. 8C), suggesting the lack of fucose on these cells. To confirm that the AAL-ve phenotype was due to the lack of functional GDP-fucose transporter gene, the enriched cells were also separately transfected with a construct that expresses the GDP-fucose transporter and analyzed by FACS. As shown in (FIG. 8D), a large number of cells became AAL-positive, indicating that the pool contained mutant cells that lack functional GDP-fucose transporter.

Single cells were then isolated from this pool into 96-well plate for further growth and characterization. Genomic DNA from each clone was extracted and the GDP-fucose transporter target locus was amplified by specific PCR primers:

(SEQ ID NO: 43)
5′-GGCGCCTCTGAAGCGGTCCAGGATCC
and
(SEQ ID NO: 44)
5′-GCCACATGTGAGCAGGGCATAGAAGG

giving a 520-bp PCR product in the case of wild-type CHO-K1 cells. PCR product is then TOPO-cloned and sequenced to identify the mutation. DNA sequencing results for the PCR products of a few selected clones showed several random deletion and insertion mutations around the ZFN target site, confirming the successful ZFN-mediated inactivation of the GDP-fucose transporter gene in these knockout mutant clones.

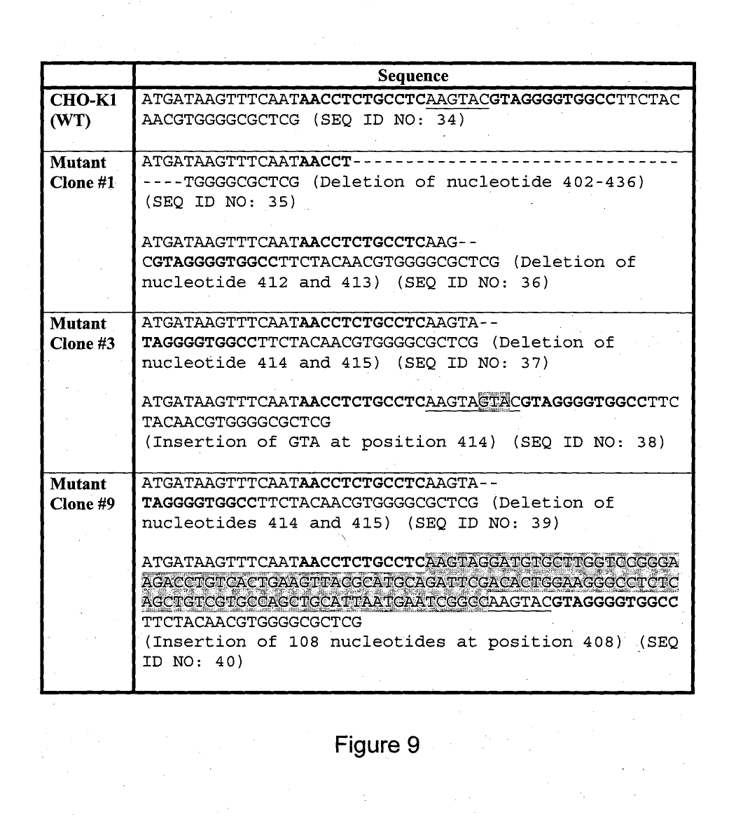

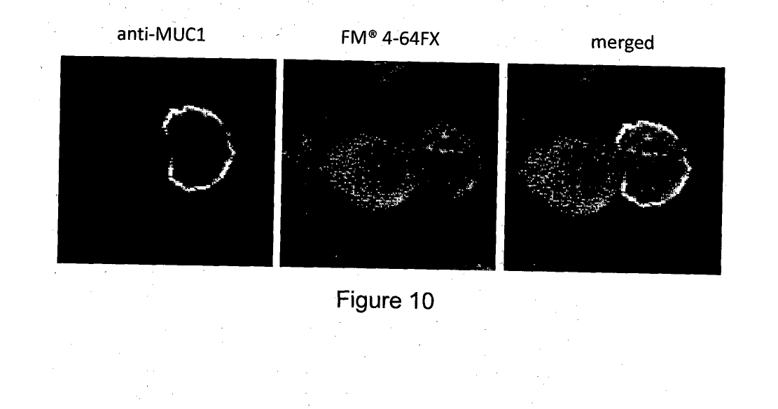

Three mutant clones were identified by this single-cell cloning process. Deletion mutations occurred in both alleles of the GDP-fucose gene in mutant clone #1. In mutant clone #3, 2 bps were deleted in one allele and 3 bps were inserted in another allele. In mutant clone #9, 2 bps were deleted in one allele while 108 bps were inserted into another allele. The sequencing results of three CHO-gmt3 clones are summarized in FIG. 9.

### Example 5

### Cellular Expression of MUC1

CHO cells were seeded on coverslips in 12 well plates, before being transformed with a construct that expresses MUC1.

Two days post transfection the cells on the coverslips were stained with anti-MUC1 antibody and a cell membrane marker FM®4-64FX for confocal microscopy. Microscopy reveals that MUC1 is expressed on the cell surface of the CHO cells.

### Example 6

### Using Cell Lysates Prepared from CHO Glycosylation Mutants Expressing Human MUC1 to Pulse Dendritic Cells as Anti-Cancer Vaccines to Treat Patients

In recent years several methods have been developed to use a patients own dendritic cells as a therapeutic anti-cancer vaccine. The dendritic cells isolated from the patient are exposed, ex vivo, to a tumor associated antigen before being returned to the patient in an attempt to elicit clinically relevant immune responses. Glycopeptides generated by the modified cells described herein may be useful in inducing anti-cancer immune responses. The dendritic cells may be exposed to a purified or isolated glycoprotein produced in a cell described herein or, alternatively, may be exposed to a lysate of such a cell which contains the glycoprotein.

### Procedure:

### Preparation of Cell Lysates:

1. Generation of CHO cell lines that express MUC1: Stable expression of human mucin 1 (MUC1) on the cell surface of CHO-K1 cells, CHO-gmt1, CHO-gmt2, CHO-gmt5 and CHO-gmt6 cells. MUC1 constructs can have variable numbers of tandem repeats, 1 to 500, for example. These recombinant cell lines can be used to treat cancer patients separately or combined.
2. The recombinant cells will be cultured in GMP conditions. For each treatment, 10 million of cells are needed. Cells will be subjected to 5 cycles of freezing (with liquid nitrogen) and thawing in water bath of 37 degrees.
3. Lysates will be centrifuged 440 g for 10 minutes, followed by 10 kg for 30 minutes. Supernatants will be kept at −80° C. for later use.

### Prepare and Pulse Dendritic Cells:

1. 200 ml of peripheral blood is collected from each patient. Peripheral blood mononuclear cells were isolated and cultured in the presence of several cytokines.
2. The cells in the culture are pulsed with the CHO cell lysates prepared as described above by adding the lysates into the culture media.
3. More cytokines are added into the culture medium.
4. Mature dendritic cells are collected by FACS sorting using dendritic markers.

Inject the cell lysate-pulsed dendritic cells into the patients:
1. 5 million dendritic cells are injected into each patient. Total 10 injections, biweekly intervals.
2. The progression of the cancer in each cancer patient is monitored and evaluated.

### REFERENCES FOR EXAMPLE 3

1. Hirschberg, C. B., Robbins, P. W., and Abeijon, C. (1998) Annu. Rev. Biochem. 67, 49-69
2. Jaeken, J. and Matthijs, G. (2007) Annu. Rev. Genomics Hum. Genet. 8, 261-278
3. Chan, K. F., Zhang, P., and Song, Z. (2010) Glycobiology 20, 689-701
4. Aoki, K., Sun-Wada, G. H., Segawa, H., Yoshioka, S., Ishida, N., and Kawakita, M. (1999) J. Biochem. 126, 940-950
5. Aoki, K., Ishida, N., and Kawakita, M. (2001) J. Biol. Chem. 276, 21555-21561
6. Aoki, K., Ishida, N., and Kawakita, M. (2003) J. Biol. Chem. 278, 22887-22893
7. Patnaik, S. K. and Stanley, P. (2006) Methods Enzymol. 416, 159-182
8. Lubke, T., Marquardt, T., Etzioni, A., Hartmann, E., von Figura, K., and Korner, C. (2001) Nat Genet. 28, 73-76
9. Luhn, K., Wild, M. K., Eckhardt, M., Gerardy-Schahn, R., and Vestweber, D. (2001) Nat Genet. 28, 69-72
10. Martinez-Duncker, I., Dupre, T., Piller, V., Piller, F., Candelier, J. J., Trichet, C., Tchernia, G., Oriol, R., and Mollicone, R. (2005) Blood 105, 2671-2676
11. Eckhardt, M., Gotza, B., and Gerardy-Schahn, R. (1999) J. Biol. Chem. 274, 8779-8787
12. Shields, R. L., Lai, J., Keck, R., O'Connell, L. Y., Hong, K., Meng, Y. G., Weikert, S. H., and Presta, L. G. (2002) J. Biol. Chem. 277, 26733-26740,
13. Shinkawa, T., Nakamura, K., Yamane, N., Shoji-Hosaka, E., Kanda, Y., Sakurada, M., Uchida, K., Anazawa, H., Satoh, M., Yamasaki, M., Hanai, N., and shitara, K. (2003) J. Biol. Chem. 278, 3466-3473
14. Kaneko, Y., Nimmerjahn, F., and Ravetch, J. V. (2006) Science 313, 670-673
15. Anthony, R. M., Nimmerjahn, F., Ashline, D. J., Reinhold, V. N., Paulson, J. C., and Ravetch, J. V. (2008) Science 320, 373-376
16. Lim, S. F., Lee, M. M., Zhang, P., and Song, Z. (2008) Glycobiology 18, 851-860
17. Sander, J. D., Zaback, P., Joung, J. K., Voytas, D. F., and Dobbs, D. (2007) Nucleic Acids Res. 35, W599-W605
18. Segal, D. J., Dreier, B., Beerli, R. R., and Barbas, C. F., III (1999) Proc. Natl. Acad. Sci. U.S.A 96, 2758-2763
19. Dreier, B., Segal, D. J., and Barbas, C. F., III (2000) J. Mol. Biol. 303, 489-502
20. Liu, Q., Xia, Z., Zhong, X., and Case, C. C. (2002) J. Biol. Chem. 277, 3850-3856
21. Urnov, F. D., Miller, J. C., Lee, Y. L., Beausejour, C. M., Rock, J. M., Augustus, S., Jamieson, A. C., Porteus, M. H., Gregory, P. D., and Holmes, M. C. (2005) Nature 435, 646-651
22. Doyon, Y., McCammon, J. M., Miller, J. C., Faraji, F., Ngo, C., Katibah, G. E., Amora, R., Hocking, T. D., Zhang, L., Rebar, E. J., Gregory, P. D., Urnov, F. D., and Amacher, S. L. (2008) Nat Biotechnol. 26, 702-708
23. Miller, J. C., Holmes, M. C., Wang, J., Guschin, D. Y., Lee, Y. L., Rupniewski, I., Beausejour, C M., Waite, A. J., Wang, N. S., Kim, K. A., Gregory, P. D., Pabo, C. O., and Rebar, E. J. (2007) Nat Biotechnol. 25, 778-785
24. Goh, J. S., Zhang, P., Chan, K. F., Lee, M. M., Lim, S. F., and Song, Z. (2010) Metab Eng 12, 360-368
25. Sander, J. D., Maeder, M. L., Reyon, D., Voytas, D. F., Joung, J. K., and Dobbs, D. (2010) Nucleic Acids Res. 38, W462-W468
26. Matsumura, K., Higashida, K., Ishida, H., Hata, Y., Yamamoto, K., Shigeta, M., Mizuno-Horikawa, Y., Wang, X., Miyoshi, E., Gu, J., and Taniguchi, N. (2007) J. Biol. Chem. 282, 15700-15708
27. Zhao, W., Chen, T. L., Vertel, B. M., and Colley, K. J. (2006) J. Biol. Chem. 281, 31106-31118
28. Abe, M., Noda, Y., Adachi, H., and Yoda, K. (2004) J. Cell Sci. 117, 5687-5696
29. Kabuss, R., Ashikov, A., Oelmann, S., Gerardy-Schahn, R., and Bakker, H. (2005) Glycobiology 15, 905-911
30. Ishikawa, H. O., Ayukawa, T., Nakayama, M., Higashi, S., Kamiyama, S., Nishihara, S., Aoki, K., Ishida, N., Sanai, Y., and Matsuno, K. (2010) J. Biol. Chem. 285, 4122-4129
31. Lu, L., Hou, X., Shi, S., Korner, C., and Stanley, P. (2010) J. Biol. Chem. 285, 36245-36254
32. Ishiguro, T., Kawai, S., Habu, K., Sugimoto, M., Shiraiwa, H., lijima, S., Ozaki, S., Matsumoto, T., and Yamada-Okabe, H. (2010) Cancer Sci. 101, 2227-2233

### REFERENCES FOR EXAMPLE 4

Zhang P, Haryadi R, Chan K F, Teo G, Goh J, Pereira N A, et al. Identification of functional elements of the GDP-fucose transporter SLC35C1 using a novel Chinese hamster ovary mutant. Glycobiology 2012; 22:897-911.

1. A glycoprotein that is expressed by a mammalian cell, wherein the mammalian cell comprises:
a) a mutated GnT I gene and a mutated CST gene;
b) a mutated GnT I gene and a mutated UGT gene; or
c) a mutated CST gene; or
d) a mutated UGT gene; or
e) a mutated GFT gene and a mutated CST gene; or
f) a mutated GFT gene.

2-35. (canceled)


SEQUENCE LISTING (45 sequences)

SEQ ID NO: 1 - protein, 20 aa, Homo sapiens
HGVTSAPDTRPAPGSTAPPA

SEQ ID NO: 2 - protein, 25 aa, Artificial Sequence
  Description of Artificial Sequence Synthetic peptide
STAPPAHGVTSAPDTRPAPGSTAPP

SEQ ID NO: 3 - DNA, 1344 bp, Cricetulus griseus
atgctgaagaagcagtctgcagggcttgtgctttggggtgctatcctctttgtgggctggaatgccctgctgctcctcttcttctggacacgcccagcccctggcaggcccccctcagatagtgctatcgatgatgaccctgccagcctcacccgtgaggtgttccgcctggctgaggacgctgaggtggagttggagcggcagcgggggctgttgcagcaaatcagggagcatcatgctttgtggagacagaggtggaaagtgcccaccgtggcccctccagcctggccccgtgtgcctgcgaccccctcaccagccgtgatccccatcctggtcattgcctgtgaccgcagcactgtccggcgctgcttggataagttgttgcactatcggccctcagctgagcatttccccatcattgtcagccaggactgcgggcacgaagagacagcacaggtcattgcttcctatggcagtgcagtcacacacatccggcagccagacctgagtaacatcgctgtgcccccagaccaccgcaagttccagggttactacaagatcgccaggcactaccgctgggcactgggccagatcttcaacaagttcaagttcccagcagctgtggtagtggaggacgatctggaggtggcaccagacttctttgagtacttccaggccacctacccactgctgagaacagacccctccctttggtgtgtgtctgcttggaatgacaatggcaaggagcagatggtagactcaagcaaacctgagctgctctatcgaacagacttttttcctggccttggctggctgctgatggctgagctgtggacagagctggagcccaagtggcccaaggccttctgggatgactggatgcgcagacctgagcagcggaaggggcgggcctgtattcgtccagaaatttcaagaacgatgacctttggccgtaagggtgtgagccatgggcagttctttgatcagcatcttaagttcatcaagctgaaccagcagttcgtgtctttcacccagttggatttgtcatacttgcagcgggaggcttatgaccgggatttccttgcccgtgtctatagtgcccccctgctacaggtggagaaagtgaggaccaatgatcagaaggagctgggggaggtgcgggtacagtacactagcagagacagcttcaaggcctttgctaaggccctgggtgtcatggatgacctcaagtctggtgtccccagagctggctaccggggcgttgtcactttccagttcaggggtcgacgtgtccacctggcacccccacaaacctgggaaggctatgatcctagctggaattag

SEQ ID NO: 4 - protein, 336 aa, Cricetulus griseus
MAQARENVSLFFKLYCLAVMTLVAAAYTVALRYTRTTAKELYFSTTAVCVTEVIKLLISVGLLAKETGSLGRFKASLSENVLGSPKELMKLSVPSLVYAVQNNMAFLALSNLDAAVYQVTYQLKIPCTALCTVLMLNRTLSKLQWVSVFMLCGGVILVQWKPAQATKVVVEQSPLLGFGAIAIAVLCSGFAGVYFEKVLKSSDTSLWVRNIQMYLSGIVVTLVGTYLSDGAEIKEKGFFYGYTYYVWFVIFLASVGGLYTSVVVKYTDNIMKGFSAAAAIVLSTIASVMLFGLQITLSFAMGALLVCISIYLYGLPRQDTTCIQQEATSKERVIGV

SEQ ID NO: 5 - protein, 395 aa, Cricetulus griseus
MAAVGVGGSAAAAGPGAVSAGALEPGSATAAHRRLKYISLAVLVVQNASLILSIRYARTLPGDRFFATTAVVMAEVLKGVTCLLLLFAQKRGNVKHLVLFLHEAVLVQYVDTLKLAVPSLIYTLQNNLQYVAISNLPAATFQVTYQLKILTTALFSVLMLNRSLSRLQWASLLLLFTGVAIVQAQQAGGGGPRPLDQNPGAGLAAVVASCLSSGFAGVYFEKILKGSSGSVWLRNLQLGLFGTALGLVGLWWAEGTAVARRGFFFGYTPAVWGVVLNQAFGGLLVAVVVKYADNILKGFATSLSIVLSTVASIRLFGFHLDPLFALGAGLVIGAVYLYSLPRSAVKAITSASASASASGPCIHQQPPGQPPPPPQLSSRGDLITEPFLPKSVLVK

SEQ ID NO: 6 - protein, 365 aa, Cricetulus griseus
MNRAPLKRSRILRMALTGGSTASEEADEDSRNKPFLLRALQIALVVSLYWVTSISMVFLNKYLLDSPSLQLDTPIFVTFYQCLVTSLLCKGLSTLATCCPGTVDFPTLNLDLKVARSVLPLSVVFIGMISFNNLCLKYVGVAFYNVGRSLTTVFNVLLSYLLLKQTTSFYALLTCGIIIGGFWLGIDQEGAEGTLSLIGTIFGVLASLCVSLNAIYTKKVLPAVDNSIWRLTFYNNVNACVLFLPLMVLLGELRALLDFAHLYSAHFWLMMTLGGLFGFAIGYVTGLQIKFTSPLTHNVSGTAKACAQTVLAVLYYEETKSFLWWTSNLMVLGGSSAYTWVRGWEMQKTQEDPSSKEGEKSAIRV

SEQ ID NO: 7 - protein, 1255 aa, Homo sapiens
MTPGTQSPFFLLLLLTVLTVVTGSGHASSTPGGEKETSATQRSSVPSSTEKNAVSMTSSVLSSHSPGSGSSTTQGQDVTLAPATEPASGSAATWGQDVTSVPVTRPALGSTTPPAHDVTSAPDNKPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDNRPALGSTAPPVHNVTSASGSASGSASTLVHNGTSARATTTPASKSTPFSIPSHHSDTPTTLASHSTKTDASSTHHSSVPPLTSSNHSTSPQLSTGVSFFFLSFHISNLQFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAATSANL

SEQ ID NO: 8 - DNA, 4139 bp, Homo sapiens
ccgctccacctctcaagcagccagcgcctgcctgaatctgttctgccccctccccacccatttcaccaccaccatgacaccgggcacccagtctcctttcttcctgctgctgctcctcacagtgcttacagttgttacaggttctggtcatgcaagctctaccccaggtggagaaaaggagacttcggctacccagagaagttcagtgcccagctctactgagaagaatgctgtgagtatgaccagcagcgtactctccagccacagccccggttcaggctcctccaccactcagggacaggatgtcactctggccccggccacggaaccagcttcaggttcagctgccacctggggacaggatgtcacctcggtcccagtcaccaggccagccctgggctccaccaccccgccagcccacgatgtcacctcagccccggacaacaagccagccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccatggtgtcacctcggccccggacaacaggcccgccttgggctccaccgcccctccagtccacaatgtcacctcggcctcaggctctgcatcaggctcagcttctactctggtgcacaacggcacctctgccagggctaccacaaccccagccagcaagagcactccattctcaattcccagccaccactctgatactcctaccacccttgccagccatagcaccaagactgatgccagtagcactcaccatagctcggtacctcctctcacctcctccaatcacagcacttctccccagttgtctactggggtctctttctttttcctgtcttttcacatttcaaacctccagtttaattcctctctggaagatcccagcaccgactactaccaagagctgcagagagacatttctgaaatgtttttgcagatttataaacaagggggttttctgggcctctccaatattaagttcaggccaggatctgtggtggtacaattgactctggccttccgagaaggtaccatcaatgtccacgacgtggagacacagttcaatcagtataaaacggaagcagcctctcgatataacctgacgatctcagacgtcagcgtgagtgatgtgccatttcctttctctgcccagtctggggctggggtgccaggctggggcatcgcgctgctggtgctggtctgtgttctggttgcgctggccattgtctatctcattgccttggctgtctgtcagtgccgccgaaagaactacgggcagctggacatctttccagcccgggatacctaccatcctatgagcgagtaccccacctaccacacccatgggcgctatgtgccccctagcagtaccgatcgtagcccctatgagaaggtttctgcaggtaacggtggcagcagcctctcttacacaaacccagcagtggcagccgcttctgccaacttgtagggcacgtcgccgctgagctgagtggccagccagtgccattccactccactcaggttcttcaggccagagcccctgcaccctgtttgggctggtgagctgggagttcaggtgggctgctcacagcctccttcagaggccccaccaatttctcggacacttctcagtgtgtggaagctcatgtgggcccctgaggctcatgcctgggaagtgttgtgggggctcccaggaggactggcccagagagccctgagatagcggggatcctgaactggactgaataaaacgtggtctcccactg

SEQ ID NO: 9 - DNA, 1209 bp, Homo sapiens
acctctcaagcagccagcgcctgcctgaatctgttctgccccctccccacccatttcaccaccaccatgacaccgggcacccagtctcctttcttcctgctgctgctcctcacagtgcttacagttgttacgggttctggtcatgcaagctctaccccaggtggagaaaaggagacttcggctacccagagaagttcagtgcccagctctactgagaagaatgctttgtctactggggtctctttctttttcctgtcttttcacatttcaaacctccagtttaattcctctctggaagatcccagcaccgactactaccaagagctgcagagagacatttctgaaatgtttttgcagatttataaacaagggggttttctgggcctctccaatattaagttcaggccaggatctgtggtggtacaattgactctggccttccgagaaggtaccatcaatgtccacgacgtggagacacagttcaatcagtataaaacggaagcagcctctcgatataacctgacgatctcagacgtcagcgtgagtgatgtgccatttcctttctctgcccagtctggggctggggtgccaggctggggcatcgcgctgctggtgctggtctgtgttctggttgcgctggccattgtctatctcattgccttggctgtctgtcagtgccgccgaaagaactacgggcagctggacatctttccagcccgggatacctaccatcctatgagcgagtaccccacctaccacacccatgggcgctatgtgccccctagcagtaccgatcgtagcccctatgagaaggtttctgcaggtaatggtggcagcagcctctcttacacaaacccagcagtggcagccacttctgccaacttgtaggggcacgtcgcccgctgagctgagtggccagccagtgccattccactccactcaggttcttcagggccagagcccctgcaccctgtttgggctggtgagctgggagttcaggtgggctgctcacagcctccttcagaggccccaccaatttctcggacacttctcagtgtgtggaagctcatgtgggcccctgagggctcatgcctgggaagtgttgtggtgggggctcccaggaggactggcccagagagccctgagatagcggggatcctgaactggactgaataaaacgtggtctcccactgcgccaaaaaaaaaaaa

SEQ ID NO: 10 - protein, 255 aa, Homo sapiens
MTPGTQSPFFLLLLLTVLTVVTGSGHASSTPGGEKETSATQRSSVPSSTEKNAFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAATSANL

SEQ ID NO: 11 - protein, 273 aa, Homo sapiens
MTPGTQSPFFLLLLLTVLTVVTGSGHASSTPGGEKETSATQRSSVPSSTEKNALSTGVSFFFLSFHISNLQFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAATSANL

SEQ ID NO: 12 - protein, 264 aa, Homo sapiens
MTPGTQSPFFLLLLLTVLTATTAPKPATVVTGSGHASSTPGGEKETSATQRSSVPSSTEKNAFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAATSANL

SEQ ID NO: 13 - protein, 255 aa, Homo sapiens; identical to SEQ ID NO: 10

SEQ ID NO: 14 - DNA, 32 bp, Cricetulus griseus
taacctctgcctcaagtacgtaggggtggcct

SEQ ID NO: 15 - DNA, 32 bp, Cricetulus griseus
aggccacccctacgtacttgaggcagaggtta

SEQ ID NO: 16 - protein, 7 aa, Artificial Sequence
  Description of Artificial Sequence Synthetic zinc finger peptide
TSGSLVR

SEQ ID NO: 17 - protein, 7 aa, Artificial Sequence
  Description of Artificial Sequence Synthetic zinc finger peptide
ERGTLAR

SEQ ID NO: 18 - protein, 7 aa, Artificial Sequence
  Description of Artificial Sequence Synthetic zinc finger peptide
RSDNLAR

SEQ ID NO: 19 - protein, 7 aa, Artificial Sequence
  Description of Artificial Sequence Synthetic zinc finger peptide
RSDALAR

SEQ ID NO: 20 - protein, 7 aa, Artificial Sequence
  Description of Artificial Sequence Synthetic zinc finger peptide
QSGSLTR

SEQ ID NO: 21 - protein, 7 aa, Artificial Sequence
  Description of Artificial Sequence Synthetic zinc finger peptide
RSDHLSR

SEQ ID NO: 22 - protein, 7 aa, Artificial Sequence
  Description of Artificial Sequence Synthetic zinc finger peptide
RSDNLAR

SEQ ID NO: 23 - protein, 7 aa, Artificial Sequence
  Description of Artificial Sequence Synthetic zinc finger peptide
QSGALAR

SEQ ID NO: 24 - DNA, 60 bp, Cricetulus griseus
atgataagtttcaataacctctgcctcaagtacgtaggggtggccttctacaacgtgggg

SEQ ID NO: 25 - DNA, 59 bp, Artificial Sequence
  Description of Artificial Sequence Synthetic Clone F deletion mutation
atgataagtttcaataacctctgcctcaagtcgtaggggtggccttctacaacgtgggg

SEQ ID NO: 26 - DNA, 59 bp, Artificial Sequence
  Description of Artificial Sequence Synthetic Clone H deletion mutation
atgataagtttcaataacctctgcctcagtacgtaggggtggccttctacaacgtgggg

SEQ ID NO: 27 - DNA, 57 bp, Artificial Sequence
  Description of Artificial Sequence Synthetic Clone E1 deletion mutation
atgataagtttcaataacctctgcctcaacgtaggggtggccttctacaacgtgggg

SEQ ID NO: 28 - DNA, 43 bp, Artificial Sequence
  Description of Artificial Sequence Synthetic Clone D deletion mutation
atgataagtttcaataacctctgtggccttctacaacgtgggg

SEQ ID NO: 29 - DNA, 33 bp, Artificial Sequence
  Description of Artificial Sequence Synthetic Clone M deletion mutation
atgataagtttcaataccttctacaacgtgggg

SEQ ID NO: 30 - DNA, 32 bp, Artificial Sequence
  Description of Artificial Sequence Synthetic Clone C1 deletion mutation
atgataagtttcaggccttctacaacgtgggg

SEQ ID NO: 31 - DNA, 20 bp, Artificial Sequence
  Description of Artificial Sequence Synthetic Clone E2 deletion mutation
atgccttctacaacgtgggg

SEQ ID NO: 32 - DNA, 64 bp, Artificial Sequence
  Description of Artificial Sequence Synthetic Clone G insertion mutation
atgataagtttcaataacctctgcctcaagtaagtacgtaggggtggccttctacaacgtgggg

SEQ ID NO: 33 - DNA, 120 bp, Artificial Sequence
  Description of Artificial Sequence Synthetic Clone C2 insertion mutation
atgataagtttcaataacctctgcctcaagtaccccattgacgtcaatgggagtttgttttggcaccaaaatcaacgggactttccaaaagtacgtaggggtggccttctacaacgtggg

SEQ ID NO: 34 - DNA, 66 bp, Cricetulus griseus
atgataagtttcaataacctctgcctcaagtacgtaggggtggccttctacaacgtggggcgctcg

SEQ ID NO: 35 - DNA, 31 bp, Artificial Sequence
  Description of Artificial Sequence Synthetic CHO-gmt3 mutant clone 1
atgataagtttcaataaccttggggcgctcg

SEQ ID NO: 36 - DNA, 64 bp, Artificial Sequence
  Description of Artificial Sequence Synthetic CHO-gmt3 mutant clone 1
atgataagtttcaataacctctgcctcaagcgtaggggtggccttctacaacgtggggcgctcg

SEQ ID NO: 37 - DNA, 64 bp, Artificial Sequence
  Description of Artificial Sequence Synthetic CHO-gmt3 mutant clone 3
atgataagtttcaataacctctgcctcaagtataggggtggccttctacaacgtggggcgctcg

SEQ ID NO: 38 - DNA, 69 bp, Artificial Sequence
  Description of Artificial Sequence Synthetic CHO-gmt3 mutant clone 3
atgataagtttcaataacctctgcctcaagtagtacgtaggggtggccttctacaacgtggggcgctcg

SEQ ID NO: 39 - DNA, 64 bp, Artificial Sequence; identical to SEQ ID NO: 37
  Description of Artificial Sequence Synthetic CHO-gmt3 mutant clone 9

SEQ ID NO: 40 - DNA, 174 bp, Artificial Sequence
  Description of Artificial Sequence Synthetic CHO-gmt3 mutant clone 9
atgataagtttcaataacctctgcctcaagtaggatgtgcttggtccgggaagacctgtcactgaagttacgcatgcagattcgacactggaagggcctctcagctgtcgtgccagctgcattaatgaatcggccaagtacgtaggggtggccttctacaacgtggggcgctcg

SEQ ID NO: 41 - DNA, 3531 bp, Homo sapiens
aaggccacgtgggggcgtgtcaggaagtgagtccagggcccgcctcccggggagtcggcctcggatgtccggaggctcctgggctgagccggcgacagagcccgggaaggcagcgagacgtgggcgccggcccagccccctcccgcgtccttcagccccaagccccgagcccctctgacccttccgcagccctccctccagccgcgcccggcctccggcagctccctgtacgcctccctccccctgcccgcccctccctcccacagccgcccatgacgccctctcggcacctcttcccactctgccacgcgtccttttcctgcaccttcgccccgcgtacctactcctgccccgccctgccattcctctcccctcccttctctctgcgacccctccctgttaggccccagcctcttctcccctcacaggtcttctctgtcctggcctcaccgccttatcctattcctctcccttgccctgtgtcttgtctcagagccccctcggggtgggagtaggttgtggagcagcacaactgggctcaccccaaagcagaacttctcaatccatgaggacaatggggaggcctttaggccagcccacatgtgacaatggagggctgcggcttccttgcggagagcacaagtgagctcactgccctggactccagggaatcagagttctggccgcggggtgacccagctcctctgctaccatgaatagggcccctctgaagcggtccaggatcctgcacatggcgctgaccggggcctcagacccctctgcagaggcagaggccaacggggagaagccctttctgctgcgggcattgcagatcgcgctggtggtctccctctactgggtcacctccatctccatggtgttccttaataagtacctgctggacagcccctccctgcggctggacacccccatcttcgtcaccttctaccagtgcctggtgaccacgctgctgtgcaaaggcctcagcgctctggccgcctgctgccctggtgccgtggacttccccagcttgcgcctggacctcagggtggcccgcagcgtcctgcccctgtcggtggtcttcatcggcatgatcaccttcaataacctctgcctcaagtacgtcggtgtggccttctacaatgtgggccgctcactcaccaccgtcttcaacgtgctgctctcctacctgctgctcaagcagaccacctccttctatgccctgctcacctgcggtatcatcatcgggggcttctggcttggtgtggaccaggagggggcagaaggcaccctgtcgtggctgggcaccgtcttcggcgtgctggctagcctctgtgtctcgctcaacgccatctacaccacgaaggtgctcccggcggtggacggcagcatctggcgcctgactttctacaacaacgtcaacgcctgcatcctcttcctgcccctgctcctgctgctcggggagcttcaggccctgcgtgactttgcccagctgggcagtgcccacttctgggggatgatgacgctgggcggcctgtttggctttgccatcggctacgtgacaggactgcagatcaagttcaccagtccgctgacccacaatgtgtcgggcacggccaaggcctgtgcccagacagtgctggccgtgctctactacgaggagaccaagagcttcctctggtggacgagcaacatgatggtgctgggcggctcctccgcctacacctgggtcaggggctgggagatgaagaagactccggaggagcccagccccaaagacagcgagaagagcgccatgggggtgtgagcaccacaggcaccctggatggcccggccccggggcccgtacacaggcggggccagcacagtagtgaaggcggtctcctggaccccagaagcgtgctgtggtgtggactgggtgctacttatagacccaatcagaatacggtggttgagaaggaaccagtgtttacaagtaatatcagaaagttgaaggaaccagtgtttacaagtaataccagaaagttgccaaacccttctctatcctctcgtatttctgagtttttgtccttcccgagggagcaccctagtgagagttgaaccccttccttctgcctccagggcctgtctgcctccacatcactctgaggacagggacaggcaacaaccttgaagggacagcaatggcaaagccacaaaggcttcactgtactcaggggagatggccctaccacagccacctggagagggttgggaagccttccgtctgcggtgggcaggcagcctcaccctgggcccacagaccggccttcctttggaggaaagtgtggcctggactgagggaggaaatgagcgagttccctctgacaccagcagatccccaggggctgctgggcagtggcctgggaatggggtggattgtgagaaagtgctcaccatctatacaccctgtatgtccagcttttgaacacaagggaaccatgcttctcttagaggttaagcagggtcattaacatcctcccccagtccctaacatcacattgtcctgcgtggctcctctggccctgagtggcacctgtccctctggtctcccagcacctggcccaggtaacagccttctgaaagcagagccaaggagctgcttctctcttctcccagttctacctccccagaagccttcctccccaggtggggctgatggagcaagggtccagactaggagccttccaccccagctgtgtctggcgcccctagatctctgcaagggaggtgttacagctggttctgagccgcttgccttgtgatggtaagacaccaacctttacattcttccctgaggttgtggctgacagagcctgcttggccccactgttagtccagcgagctcctatatcaaaatgccgtaggccgggtggcttacaaacaacagaaacgtattgctcacagtcctggggggctgggacgcccaagatcaagaggccagcagattcggactccgctgagggctgtttcccgatccatagatggtgccttctcgctgtatcctcaatggtagaagcacaaacaagcaagctccttcctgcctcttttataaggactccaaccctgttcatgagggttccgcccccatgacccaatcagctccaaaggccccacctcctaatactgtcaccttgggggtgagaattccaatgtgaatttgcagggggagtgggggacacacacaaatttcggggccataccacccttcaccacaccctcctgcgctcagggtggcttgcagtccctggcccttctggtgggcatttggtatgtcctttctcttggggtgatttctgatgtttttactctatatagtgaaaagctagggagagcgggtcttctcccccctccctctccagtcccctcacaatcccagatgggttctaatgcagctgctggggcctgatgccctgagttgtttgtgattcaataaagaatccataagaaaaaaaaaaaaaaaaaaaaaaaaaaaaaaaaaaaaaaaaaaaa

SEQ ID NO: 42 - DNA, 30 bp, Artificial Sequence
  Description of Artificial Sequence Synthetic ideal target sequence for 4-fingered ZFNs
nncnncnncnncnnnnnngnngnngnngnn

SEQ ID NO: 43 - DNA, 26 bp, Artificial Sequence
  Description of Artificial Sequence Synthetic primer
ggcgcctctgaagcggtccaggatcc

SEQ ID NO: 44 - DNA, 26 bp, Artificial Sequence
  Description of Artificial Sequence Synthetic primer
gccacatgtgagcagggcatagaagg

SEQ ID NO: 45 - protein, 6 aa, Artificial Sequence
  Description of Artificial Sequence Synthetic 6xHis tag
HHHHHH